(19) United States
(12) Patent Application Publication
(10) Pub. No.: US 2026/0341432 A1
(43) Pub. Date: Oct. 29, 2026

(54) SYSTEM

(71) Applicant: SoftBank Group Corp., Tokyo (JP)

(72) Inventor: Hidehiko AIHARA, Tokyo (JP)

(73) Assignee: SoftBank Group Corp., Tokyo (JP)

(21) Appl. No.: 19/649,202

(22) Filed: Apr. 16, 2026

(30) Foreign Application Priority Data
Apr. 23, 2025 (JP) 2025-071614

(51) Int. Cl. G16H 10/60; G06F 40/295; H04L 9/00

(52) U.S. Cl. CPC G16H 10/60; G06F 40/295; H04L 9/50

(57) ABSTRACT

The present invention relates to a medical information system comprising a medical data management unit, an AI diagnosis transparency unit, a patient explanation generation unit, an inter-hospital data linkage unit, and a falsification detection unit, and integrally realizing chronological recording of medical data and falsification prevention by a hash chain, automatic generation of a diagnosis basis and explanatory text output by AI, automatic generation of an explanatory text easy for a patient to be convinced of, summary generation of a medical history, secure data sharing between medical institutions by encrypting the medical data, and fraud detection of the medical data by utilizing generative AI as an essential element, and is characterized by dramatically improving transparency, reliability, explainability, safety, and efficiency of the medical data in a medical field.

### CROSS-REFERENCE TO RELATED APPLICATION

The present application claims priority to and incorporates by reference the entire contents of Japanese Patent Application No. 2025-071614 filed in Japan on Apr. 23, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

The problem to be solved by the present invention is the difficulty in appropriately utilizing a medical history of a patient due to medical data being fragmented for each medical institution in a medical field, the problem of reliability that it is extremely difficult to detect fraud when medical information such as an electronic medical record or health checkup data is falsified, furthermore, the problem of transparency and explainability that a medical worker or a patient cannot sufficiently trust an AI diagnosis because a diagnosis basis of “why the diagnosis result was obtained” is opaque in diagnosis support using AI, and social implementation and spread of the AI diagnosis do not progress, in addition, the problem of patient satisfaction that an easy-to-understand explanation for the patient to be convinced of their own treatment policy and receive treatment with peace of mind is not sufficiently provided, and the problem of delay and inefficiency of optimal medical provision due to a lack of a mechanism for securely and efficiently linking and sharing medical data between medical institutions, and an object thereof is to comprehensively and simultaneously solve these problems.

### SUMMARY OF THE INVENTION

A means for solving the problem according to the present invention is to configure a system comprising a medical data management unit, an AI diagnosis transparency unit, a patient explanation generation unit, an inter-hospital data linkage unit, and a falsification detection unit. That is, the medical data management unit records medical data for each patient in chronological order, assigns patient identification information, medical content, a diagnosis result, a diagnosis basis, a recording time, and a hash value of immediately preceding medical data to each medical data, and stores the medical data as a hash chain, thereby realizing history management and falsification prevention of the medical data. The AI diagnosis transparency unit performs a diagnosis based on the medical data using generative AI, and explicitly generates a data item, a diagnosis logic, and a diagnosis basis used for the diagnosis as an explanatory text, thereby ensuring transparency and explainability of the AI diagnosis. The patient explanation generation unit automatically generates a treatment policy explanatory text in a natural language easy for a patient to understand using generative AI based on the diagnosis result and the diagnosis basis generated by the AI diagnosis transparency unit, thereby aiming to improve a sense of conviction and a sense of security for the patient. The inter-hospital data linkage unit encrypts the medical data recorded by the medical data management unit to enable secure sharing with another medical institution, and enables a receiving side to decrypt the medical data to view a medical history, thereby realizing secure and efficient data linkage between medical institutions. The falsification detection unit automatically detects presence or absence of falsification of the medical data by verifying the hash chain, thereby ensuring reliability of medical information. By these units cooperating and operating, it is possible to comprehensively and simultaneously solve conventional problems such as siloing of medical data, falsification risk, opacity of AI diagnosis, insufficiency of patient explanation, and inefficiency of inter-hospital data linkage.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor indicated by reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), among others.

In the following embodiments, a RAM (Random Access Memory) indicated by reference numeral is a memory where information is temporarily stored and used as a work memory by the processor.

In the following embodiments, a storage indicated by reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) indicated by reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

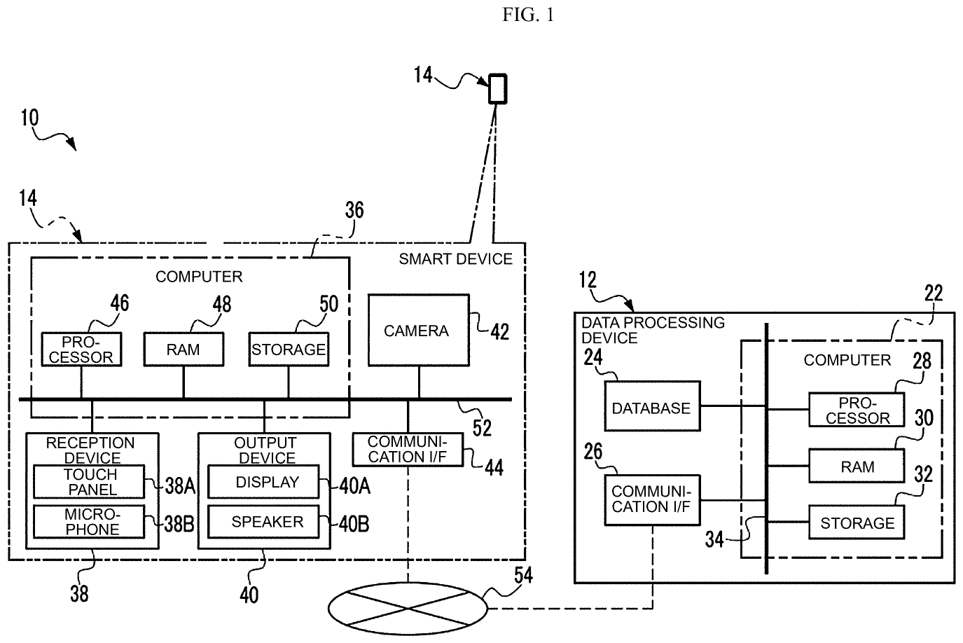

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, camera 42, and communication I/F 44 are also connected to the bus 52.

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A in the processor 46 sends data indicating user input accepted by the touch panel 38A and microphone 38B to the data processing device 12. In the data processing device 12, the specific processing unit 290 acquires data indicating user input.

The output device 40 comprises a display 40A and a speaker 40B, among others, and presents data to the user 20 by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

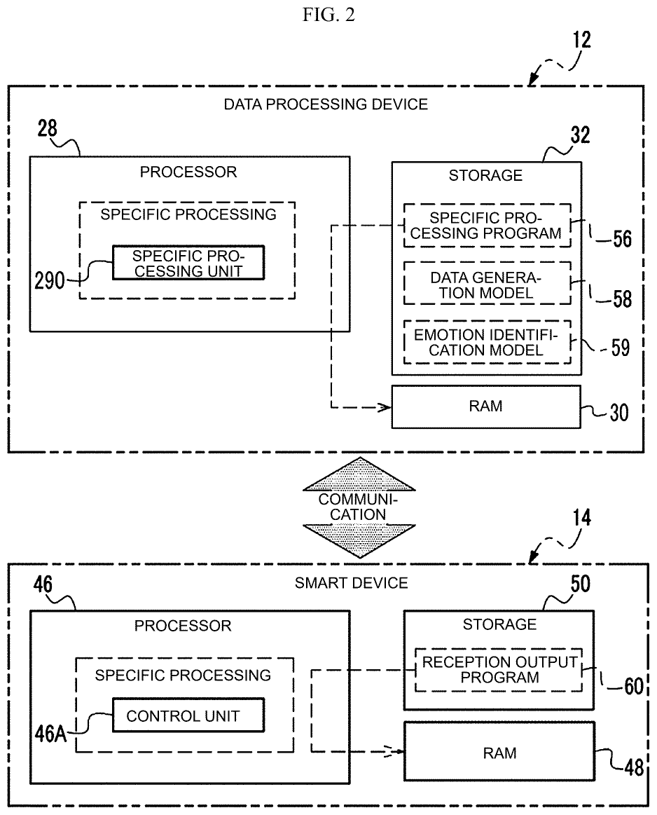

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, and various estimations and predictions related to the user's emotions are performed, but it is not limited to such examples. Furthermore, emotion estimation and prediction may include, for example, emotion analysis.

In the smart device 14, reception output processing is performed by the processor 46. The storage 50 stores a reception output program 60. The reception output program 60 is used in conjunction with the specific processing program 56 by the data processing system 10. The processor 46 reads the reception output program 60 from the storage 50 and executes it on the RAM 48. The reception output processing is realized by the processor 46 operating as a control unit 46A according to the reception output program 60 executed on the RAM 48. The smart device 14 may also have similar data generation models and emotion identification models as the data generation model 58 and emotion identification model 59, and perform the same processing as the specific processing unit 290 using these models. The reception output processing is realized by the processor 46 operating as the control unit 46A according to the reception output program 60 executed on the RAM 48.

Other devices besides the data processing device 12 may have the data generation model 58.

For example, a server device (e.g., a generation server) may have the data generation model 58.

In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example 1

The flow of the specific processing in Example 1 will be described. Each part of the system described below is realized by the data processing device 12 and the smart device 14. Additionally, the data processing device 12 is referred to as a “server,” and the smart device 14 is referred to as a “terminal.”

### Embodiment for Carrying out the Present Invention

Embodiments of the present invention will be described more specifically and in detail. The system of the present invention comprises a medical data management unit, an AI diagnosis transparency unit, a patient explanation generation unit, an inter-hospital data linkage unit, and a falsification detection unit, and the functions of these units are shared by either a server or a terminal, or both. Hereinafter, the configuration of each unit, processing procedures, a method of utilizing generative AI, role sharing between the server and the terminal, and specific usage examples will be comprehensively described. Specifically, the present system adopts a distributed computing architecture. The present system utilizes a parallel computing cluster composed of a plurality of computing nodes. The present system containerizes each processing unit as a microservice. The present system dynamically allocates processing requests to each container via a load balancer. The present system thereby dramatically improves processing speed. The present system integrally operates a plurality of machine learning models such as a large language model and a convolutional neural network. The present system executes inference processing of each model in parallel on a GPU memory. The present system thereby minimizes inference delay. The present system executes unconventional data processing based on feature extraction and probabilistic inference in a high-dimensional vector space, rather than mere automation of human work. The present system thereby significantly improves analysis accuracy of medical data compared to conventional methods. The present system transmits and receives serialized tensor format data using protocol buffers in data communication between the respective processing units. The present system thereby suppresses consumption of a communication band. As a technical effect, the present system achieves both real-time processing of a vast amount of medical data and highly accurate diagnosis support. As a specific application field, the present system functions as an integrated medical information system in a large-scale general hospital or a base system in a wide-area medical linkage network.

The medical data management unit records and manages medical data for each patient in chronological order using a database system constructed on a server. For example, patient identification information (patient ID, name, date of birth, etc.), medical content (medical examination, test, prescription, surgery, etc.), vital data (blood pressure, body temperature, pulse, blood glucose level, etc.), a diagnosis result, a diagnosis basis, a recording time, and the like are input from an outpatient reception terminal or a tablet terminal for nurses. These data are centrally managed on the server side, and a hash value of immediately preceding medical data is assigned to each medical data, whereby the medical data is stored as a hash chain. For example, when a patient N undergoes a blood test in the morning and a doctor's examination in the afternoon, the respective medical data are recorded independently, and the hash value of the medical data in the morning is assigned to the medical data in the afternoon. Furthermore, even when a patient O visits a plurality of medical institutions, the medical data of each medical institution are integrated and managed as a unified medical history of the patient O. Specifically, the present medical data management unit structures various input medical data as a multidimensional time-series tensor. The present medical data management unit converts the medical content described in a natural language into a 768-dimensional dense vector using an embedding layer of a large language model. The present medical data management unit normalizes the vital data and holds it as an array of floating-point numbers. The present medical data management unit combines these vectors and arrays to generate an integrated feature tensor for each patient. The present medical data management unit stores the generated tensor in a distributed key-value store. The present medical data management unit calculates a cryptographic hash value of an immediately preceding data block using a SHA256 algorithm at the time of data storage. The present medical data management unit embeds the calculated hash value in a header of a current data block. The present medical data management unit makes falsification of data mathematically detectable by this hash chain structure. The present medical data management unit aligns data asynchronously transmitted from a plurality of medical institutions by a merge sort algorithm based on a time stamp. The present medical data management unit ensures data consistency by this processing. As a technical effect, the present medical data management unit simultaneously ensures completeness and availability of medical data in a distributed environment. As a specific application field, the present medical data management unit is used in a wide-area backup system for electronic medical records or a patient information sharing platform among a plurality of hospitals.

The AI diagnosis transparency unit is realized by a generative AI engine operating on the server. For example, when blood test data of a patient P (blood glucose level 220 mg/dL, HbA1c 8.0%, cholesterol 250 mg/dL) is input, the generative AI analyzes these data and automatically generates a plurality of diagnosis results and bases thereof, such as “the possibility of diabetes is high because the blood glucose level exceeds 200 mg/dL and HbA1c also exceeds a reference value” and “there is a risk of dyslipidemia because the cholesterol level is high”. Furthermore, the generative AI also refers to specific data items used for the diagnosis, medical diagnostic criteria, past medical history, family history, lifestyle information, and the like, and outputs a more detailed and multifaceted diagnosis basis as an explanatory text, such as “a chronic hyperglycemic state is suspected because the blood glucose level has shown a high value in the past three tests as well” and “genetic risk was also considered because there is a diabetic patient in the family”. The generative AI can also generate a plurality of patterns of the explanatory text of the diagnosis basis according to the application, such as for medical workers, for patients, and for insurance examination. Specifically, the present AI diagnosis transparency unit comprises an autoregressive large language model based on a transformer architecture. The present AI diagnosis transparency unit receives, as input data, a numerical vector of a blood test of a patient, a time-series array representing a past medical history, and an embedding vector of categorical data including a family history. The present AI diagnosis transparency unit acquires, for example, a 128-dimensional numerical vector obtained by normalizing biological indicators such as a blood glucose level and a cholesterol level as the input data. The present AI diagnosis transparency unit also acquires, as the input data, a 512-dimensional time-series array obtained by vectorizing medical records for the past five years by natural language processing. The present AI diagnosis transparency unit further combines these input vectors into a single context tensor that can be input to a self-attention mechanism as preprocessing. The present AI diagnosis transparency unit supplies this context tensor to an input layer of the large language model. The present AI diagnosis transparency unit calculates a hidden correlation between respective feature amounts in a high-dimensional space in a multi-layer transformer block of the large language model. The present AI diagnosis transparency unit generates, as output data, a predicted probability distribution of a disease, an importance score of a feature amount serving as a diagnosis basis, and a token sequence of an explanatory text in a natural language. The present AI diagnosis transparency unit outputs, for example, a score setting the predicted probability of diabetes to 0.92 as the output data. The present AI diagnosis transparency unit also outputs, as the output data, an attention weight setting the importance of the blood glucose level to 0.85. The present AI diagnosis transparency unit further outputs, as the output data, a structured text including specialized medical terms for medical workers. The present AI diagnosis transparency unit transfers this output result as an input to the subsequent patient explanation generation unit. The present AI diagnosis transparency unit also executes branching processing for activating an emergency alert module when the predicted probability exceeds a predetermined threshold. In learning of the model, the present AI diagnosis transparency unit performs pre-training using millions of anonymized electronic medical record data and a medical paper dataset. The present AI diagnosis transparency unit further executes fine-tuning using reinforcement learning so as to imitate a diagnosis process of a specialist. The present AI diagnosis transparency unit uses a composite loss function combining a cross-entropy loss and a reward function based on an evaluation score of a specialist as an error function. The present AI diagnosis transparency unit optimizes weights of a network using a gradient descent method. As a technical effect, the present AI diagnosis transparency unit visualizes an inference process of a deep learning model that is likely to be a black box, and dramatically improves the reliability of AI in a medical field. As a specific application field, the present AI diagnosis transparency unit is utilized as a diagnosis support system in a regional medical institution lacking specialists or an educational simulator for young doctors.

The patient explanation generation unit automatically generates an explanatory text in a natural language easy for a patient to understand based on the diagnosis result and the diagnosis basis received by the generative AI engine on the server from the AI diagnosis transparency unit. For example, when a patient Q is diagnosed with hypertension, an explanatory text such as “Your blood pressure is 150/95 mmHg, which exceeds the reference value (140/90 mmHg), so you have been diagnosed with hypertension. From now on, salt reduction, exercise, and taking antihypertensive drugs as necessary are recommended.” is generated. Furthermore, the generative AI considers the patient's age, gender, past medical history, comprehension level, language setting, and the like, and generates an explanatory text including lifestyle guidance, for example, for an elderly person, such as “If high blood pressure continues, the risk of stroke and heart disease increases, so please do not forget to measure your blood pressure and take your medicine every day.” In the case of a pediatric patient, an explanatory text is generated for a guardian, such as “Because your child's blood glucose level is high, it is necessary to pay attention to diet and exercise from now on and undergo regular examinations.” The explanatory text is generated on the server side and utilized in various ways such as displaying, printing, sending an e-mail, and posting on a patient portal site on a doctor terminal or a patient terminal (smartphone, tablet, personal computer, etc.). Specifically, the present patient explanation generation unit utilizes an encoder-decoder type neural network specialized for conditional text generation. The present patient explanation generation unit receives, as input data, a probability distribution of a diagnosis result output from the AI diagnosis transparency unit, an importance score of a feature amount serving as a basis, and a profile vector including patient attribute information. The present patient explanation generation unit receives, for example, numerical data indicating a diagnosis probability of hypertension of 0.95 as the input data. The present patient explanation generation unit also receives, as the input data, a 64-dimensional attribute vector encoding the patient's age and health literacy score. As preprocessing, the present patient explanation generation unit embeds these input data in a prompt template, tokenizes them, and inputs them to a model. The present patient explanation generation unit searches for an optimal combination of words using a beam search algorithm in a decoder network. The present patient explanation generation unit generates, as output data, a text string in a natural language optimized for the patient's comprehension level, and a metadata tag for emphasizing an important part. The present patient explanation generation unit outputs, for example, a text in which technical terms are replaced with plain expressions as the output data. The present patient explanation generation unit also outputs, as the output data, structured data including an HTML tag for displaying a numerical value of blood pressure in red. The present patient explanation generation unit transmits this output result to a subsequent distribution module. The present distribution module executes branching processing for dynamically adjusting the length of the text according to the screen size of the terminal. In learning of the model, the present patient explanation generation unit performs supervised learning using a corpus of plain explanatory texts created by a medical communication expert. The present patient explanation generation unit applies reinforcement learning using an automatic evaluation index for evaluating the readability of the generated text as a reward. As a technical effect, the present patient explanation generation unit automates the provision of individualized medical information for each patient, and significantly reduces the workload related to informed consent of a doctor. As a specific application field, the present patient explanation generation unit is used as an automatic follow-up system in an online medical treatment platform or a personalization function of a health management application for patients.

The inter-hospital data linkage unit has a function of encrypting medical data on the server side and enabling secure sharing with another medical institution. For example, when a patient R transfers to another hospital, a server of a transfer source medical institution encrypts a medical history of the patient R and transmits it to a server of a transfer destination medical institution via a secure communication path (VPN, TLS, etc.). In the transfer destination medical institution, the medical history of the patient R can be decrypted on the server side and integrated into its own electronic medical record system. Furthermore, when a patient S is receiving treatment at a plurality of medical institutions, servers of the respective medical institutions mutually encrypt and share medical data, and the entire medical history of the patient S can be comprehensively grasped. For example, at the time of emergency transport, an emergency hospital can immediately acquire the medical history of the patient S and determine an optimal treatment policy by referring to a past medical history, allergy information, and latest prescription drug information. Encryption/decryption processing is executed on the server side, and viewing and inputting of decrypted data are performed on the terminal side. Specifically, the present inter-hospital data linkage unit executes a hybrid encryption algorithm combining a public key cryptosystem and a common key cryptosystem. The present inter-hospital data linkage unit encrypts a medical data tensor to be transmitted at high speed using an AES 256-bit algorithm. The present inter-hospital data linkage unit encrypts a common key used at that time with a public key of a receiving side medical institution using an RSA 4096-bit algorithm. The present inter-hospital data linkage unit combines the encrypted data payload and the encrypted common key to generate a secure data packet. The present inter-hospital data linkage unit transmits this data packet via a mutually authenticated TLS tunnel. The present inter-hospital data linkage unit executes decryption processing using a secret key in a hardware security module on the receiving side. The present inter-hospital data linkage unit physically prevents leakage of the key on a memory by this processing. The present inter-hospital data linkage unit further applies a differential privacy technology to the shared data. The present inter-hospital data linkage unit adds Laplace noise to the data, thereby maintaining statistical usefulness while making it difficult to identify an individual. As a technical effect, the present inter-hospital data linkage unit maximizes resistance to data interception and man-in-the-middle attacks via a network, and realizes secure wide-area sharing of sensitive medical information. As a specific application field, the present inter-hospital data linkage unit functions as an essential base technology in a national-scale electronic health record network or construction of an integrated database across a plurality of independent medical corporations.

The falsification detection unit verifies a hash chain of medical data on the server side, and automatically detects presence or absence of falsification. For example, when an unauthorized rewriting is performed on a medical history of a patient T, a falsified part is specified by hash chain verification, and a warning is notified to an administrator or a medical worker. Furthermore, the falsification detection unit analyzes whether an unnatural change or an abnormal pattern is included in the content of the medical data using generative AI. For example, when a part of a medical record is different from a normal medical practice, when a plurality of medical data are changed collectively in a short period of time, or when there is a contradiction between medical content and a diagnosis result, a pattern with a high possibility of falsification or fraud is automatically detected, and warning information is generated and notified. For example, in a medical record of a patient U, when diagnosis results by a plurality of different doctors are redundantly recorded on the same day, or when past medical data suddenly disappears, the generative AI detects an abnormal pattern and transmits an alert to a system administrator. Specifically, the present falsification detection unit adopts a hybrid verification architecture combining recalculation of a cryptographic hash function and a deep anomaly detection model. As regular batch processing, the present falsification detection unit recalculates hash values of all records in a database and collates them with a stored hash chain. The present falsification detection unit reliably finds even a 1-bit data mutation by this deterministic rule-based processing. The present falsification detection unit further executes semantic anomaly detection using an autoencoder-type neural network. The present falsification detection unit receives, as input data, a multidimensional feature tensor representing a time-series change of a medical record. The present falsification detection unit acquires, for example, a 256-dimensional tensor including a data update frequency, an authority level of an updater, and a vector representation of medical content as the input data. As preprocessing, the present falsification detection unit normalizes these data and generates a time-series sequence by a sliding window method. The present falsification detection unit inputs this sequence to an encoder network of an autoencoder and compresses it into a low-dimensional latent space. The present falsification detection unit reconstructs original input data from a latent representation using a decoder network. The present falsification detection unit outputs a reconstruction error score between the input data and the reconstructed data as output data. The present falsification detection unit outputs, for example, an error score that is less than 0.01 in the case of a normal data update as the output data. The present falsification detection unit also outputs, as the output data, a high error score that becomes 0.85 when an abnormal collective change is performed. The present falsification detection unit transmits this output result to a subsequent threshold determination module. The present threshold determination module executes branching processing for isolating a corresponding record and displaying an emergency alert on a dashboard of a system administrator when the error score exceeds a dynamically set threshold. In learning of the model, the present falsification detection unit performs unsupervised learning using only update history data of normal medical records. As a technical effect, the present falsification detection unit detects not only simple data falsification but also malicious data manipulation using legitimate authority and advanced fraud including logical contradiction in advance, and ensures the evidence capacity of medical data. As a specific application field, the present falsification detection unit is utilized in an authenticity proof system for electronic medical records in medical lawsuits or a fraudulent billing prevention system for medical insurance.

Furthermore, generative AI is also utilized for generating a summary of medical data. For example, when a medical history of a patient V for the past 10 years reaches hundreds of cases, the generative AI automatically summarizes a main diagnosis/treatment progress, and generates a summary text such as “continued hypertension treatment for the past 10 years, diagnosed with diabetes 5 years ago, hospitalized for myocardial infarction 3 years ago, currently using insulin treatment and antihypertensive drugs together”. Thereby, a medical worker can grasp the entire picture of the patient V in a short time. The summary text is generated on the server side and can be viewed on a doctor terminal or a nurse terminal. Specifically, the present system executes pipeline processing combining extractive summarization and generative summarization. The present system first divides text data of hundreds of medical histories into sentence units, and maps each sentence to a high-dimensional vector space using a pre-trained language model. The present system applies a PageRank algorithm based on graph theory to construct a graph network in which a cosine similarity between vectors is used as an edge weight. The present system specifies a medically important event by extracting a sentence having high centrality on this graph. The present system next configures a set of extracted important sentences as an input prompt of the generative AI. The present system receives, as input data, a token sequence of important sentences arranged in chronological order, and a control parameter designating the length of a summary. The present system acquires, for example, a text sequence of up to 4096 tokens including past diagnosis names, prescription drug change histories, and surgery records as the input data. The present system autoregressively generates a smooth summary text retaining a context using a transformer-based decoder network. The present system outputs, as output data, a summary text that fits within a designated number of characters, and linkage metadata indicating which part of an original medical record each summary text is derived from. The present system outputs, for example, a summary text of about 300 characters and an ID list of original medical records serving as a basis thereof as the output data. The present system transmits this output result to a subsequent user interface drawing module. The present drawing module generates an interactive screen that pops up an original detailed medical record when a specific disease name in the summary text is clicked. In learning of the model, the present system performs fine-tuning using a dataset of summary texts created by a specialist. As a technical effect, the present system dramatically reduces the time for a human to read a vast amount of past medical records, and supports rapid decision-making under a situation with severe time constraints such as emergency medical care. As a specific application field, the present system is used in a summary creation system integrating a medical questionnaire of a first-visit patient and past medical records, or an automatic analysis system for a letter of introduction from another hospital.

As an implementation example of each unit of the system on a server/terminal, the medical data management unit, the AI diagnosis transparency unit, the inter-hospital data linkage unit, and the falsification detection unit are mainly implemented on the server side. Thereby, processing with a high calculation load such as centralized management of a vast amount of medical data, advanced AI processing, encryption/decryption processing, and hash chain verification can be centrally executed on the server. On the other hand, the patient explanation generation unit generates an explanatory text on the server side, and is utilized as a user interface such as displaying, printing, and transmitting the explanatory text on the terminal side (doctor terminal, patient terminal, tablet, smartphone, etc.). For example, a doctor can explain a treatment policy while simultaneously viewing a medical history, an AI diagnosis result, and an explanatory text of a patient W on a terminal in an examination room, or a patient can confirm his/her own diagnosis result and explanatory text on a smartphone at home. Specifically, the present system adopts a hybrid processing architecture in which a cloud computing environment and an edge device are linked. The present system constructs a cluster of high-performance computing nodes equipped with a plurality of GPUs on the server side. The present system dynamically scales computing resources according to a load of AI inference or encryption processing using a container orchestration tool. The present system thereby maintains stable response performance even for simultaneous access from thousands of terminals. The present system implements a user interface as a single-page application operating on a web browser on the terminal side. The present system offloads all heavy arithmetic processing to the server side in order to minimize a processing load on the terminal side. The present system establishes a bidirectional real-time communication channel using a WebSocket protocol in communication between the server and the terminal. The present system push-distributes text data of an explanatory text generated on the server side and data for drawing a graph of a diagnosis result to the terminal as a lightweight payload in a JSON format. The present system updates a virtual DOM on a memory on the terminal side, and executes redrawing of a screen at high speed. As a technical effect, the present system uniformly provides advanced AI functions and secure data management to all users without depending on hardware performance of a terminal. As a specific application field, the present system functions as SaaS-type electronic medical record provision to a small-scale clinic with limited resources, or a back-end system of a personal health record application for a patient's personal smartphone.

Also, for example, in the case of remote medical treatment, a patient inputs medical data using a terminal from home, AI diagnosis and explanatory text generation are performed on the server side, and the result is returned to the terminal, whereby the patient can receive a convincing explanation even from a remote location. For example, in a regional medical network in which a plurality of medical institutions cooperate, it is possible to access a server from a terminal of each medical institution and share/view a medical history and an AI diagnosis result of a patient X in real time. Furthermore, a patient can confirm his/her own medical history and AI diagnosis result with a smartphone application, and share them with a family member or a caregiver as necessary. Specifically, the present system comprises a secure API gateway for supporting remote medical treatment. The present system continuously streams biological data acquired from a camera or a wearable device mounted on a patient's terminal to a server via a RESTful API. The present system receives, as input data, time-series waveform data of a heart rate, a numerical array of blood oxygen saturation, and text data of subjective symptoms input by a patient. The present system acquires, for example, a one-dimensional tensor of a heart rate sampled at 1-second intervals from a smart watch as the input data. The present system integrates these multimodal data on the server side, and inputs them to a long short-term memory network which is a time-series prediction model. The present system outputs, as output data, a probability score indicating a risk of condition deterioration after several hours, and a text of a recommended action guideline. The present system outputs, for example, a score of an arrhythmia occurrence risk of 0.85 as the output data. The present system transmits this output result to a subsequent notification module. The present notification module executes branching processing for transmitting a push notification to a patient's terminal and an attending doctor's terminal when the risk score exceeds a threshold. The present system further provides a function in which a patient himself/herself finely sets and manages data viewing authority for a family member or a caregiver using an access control list applying a blockchain technology. As a technical effect, the present system eliminates geographical constraints and enables proactive medical intervention based on real-time biological data. As a specific application field, the present system functions extremely effectively in a home monitoring system for chronic disease patients or an online medical treatment platform in a depopulated area.

As described above, the embodiment of the present invention integrally and specifically realizes various functions required in a medical field, such as recording/management/falsification prevention of medical data, diagnosis and basis explanation by AI, patient explanatory text generation, secure inter-hospital data linkage, falsification detection, and medical data summarization, with generative AI as an essential element, and achieves both high security and convenience by role sharing between a server and a terminal, and is an extremely practical and highly expandable system that can be widely utilized in all medical scenes such as outpatient medical treatment, inpatient medical treatment, emergency medical care, remote medical treatment, regional medical linkage, and self-care by a patient himself/herself. Specifically, the present system functions as an integrated information processing platform in which a data pipeline, an inference engine of a machine learning model, and an encrypted communication infrastructure are tightly coupled, rather than a mere collection of individual software tools. The present system shares a standardized data schema among respective modules, and realizes loosely coupled linkage based on a microservice architecture. The present system thereby facilitates addition of a new AI model and linkage expansion with an external medical database in the future. The present system incorporates an observability tool for monitoring an operating status of the entire system, and measures inference accuracy and processing delay of each AI model in real time. The present system includes a mechanism of MLOps for automatically triggering a retraining pipeline of a model based on measured metrics. As a technical effect, the present system constructs a self-evolving system that securely and intelligently manages the entire life cycle of medical data, and continuously improves the quality of medical services for patients while reducing the cognitive load of medical workers. As a specific application field, the present system is widely deployed as a core operating system in a next-generation smart hospital concept or a base infrastructure for promoting national-level medical digital transformation.

### System Configuration

The system according to the present embodiment comprises a medical data management unit, an AI diagnosis transparency unit, a patient explanation generation unit, an inter-hospital data linkage unit, and a falsification detection unit. The medical data management unit has a function of recording and managing medical data for each patient in chronological order, and stores, for example, patient identification information, medical content, vital data, a diagnosis result, a diagnosis basis, a recording time, and the like input from an outpatient reception terminal or a tablet terminal for nurses in a database on a server. When a patient undergoes a blood test in the morning and a medical examination by a doctor in the afternoon, the medical data management unit can independently record each medical data, assign a hash value of the medical data of the morning to the medical data of the afternoon, and store the medical data as a hash chain. In addition, the medical data management unit can integrate medical data of a patient who has visited a plurality of medical institutions, and manage a unified medical history for each patient. Furthermore, the medical data management unit records multifaceted data including a past medical history, a family history, lifestyle habit information, and the like of the patient, and can manage, for example, a surgery history, allergy information, a medication history, and the like that the patient has received in the past in chronological order. The medical data management unit automatically checks whether there is any deficiency or contradiction when the medical data is input, and can issue a warning, for example, when diagnosis results by a plurality of different doctors are redundantly recorded on the same day, or when there is a contradiction between the medical content and the diagnosis result. Specifically, the medical data management unit adopts a polyglot persistence architecture combining a relational database and a graph database. The medical data management unit stores structured vital data and patient attributes in a table of the relational database, and expresses the past medical history and the family history having complex relationships as nodes and edges of the graph database. The medical data management unit implements a rule engine for verifying the input data in real time. The medical data management unit receives a JSON object of a newly registered medical record as input data. The medical data management unit acquires, for example, a data structure including a doctor ID, a patient ID, an ICD-coded diagnosis name, and a code of a prescription drug as the input data. The medical data management unit performs schema verification of the input data as preprocessing, and detects a missing value and an abnormal value. The medical data management unit further evaluates semantic consistency between the text of the medical content and the code of the diagnosis result using a natural language processing model. The medical data management unit outputs a Boolean value indicating validity of the data and a character string of an error message when a contradiction exists as output data. The medical data management unit outputs, for example, a flag indicating that the validity is false as the output data. The medical data management unit also outputs a warning text “The prescribed drug does not match the standard treatment guidelines for the diagnosed disease” as the output data. The medical data management unit transmits this output result to a subsequent transaction management module. When the validity is false, the transaction management module executes a branching process of rolling back writing to the database and prompting an input terminal to re-input. As a technical effect, the medical data management unit thoroughly performs data cleansing at an input stage and eliminates noise in subsequent AI analysis, thereby raising inference accuracy of the entire system. As a specific application field, the medical data management unit is responsible for data quality assurance in a large-scale clinical trial data collection system or a comprehensive electronic medical record system across a plurality of clinical departments.

The AI diagnosis transparency unit has a function of performing a diagnosis based on the medical data using generative AI, and explicitly outputting a diagnosis basis thereof as an explanatory text. The AI diagnosis transparency unit receives, for example, blood test data (blood glucose level, HbA1c, cholesterol level, etc.) of a patient as an input, gives a prompt text such as “Based on the blood test data of this patient, please explain possible diseases and bases thereof in detail medically” to the generative AI, and causes the generative AI to generate a diagnosis result and a basis explanation. The AI diagnosis transparency unit also refers to a specific data item, a medical diagnosis criterion, a past medical history, a family history, lifestyle habit information, and the like used for the diagnosis, and outputs a detailed and multifaceted diagnosis basis as an explanatory text, such as “the possibility of diabetes is high because the blood glucose level exceeds 200 mg/dL and HbA1c also exceeds a reference value”, “a chronic hyperglycemic state is suspected because the blood glucose level has shown a high value in the past three tests”, and “a genetic risk is also considered because there is a diabetic patient in the family”. The AI diagnosis transparency unit can also generate a plurality of patterns of the explanatory text of the diagnosis basis according to an application such as for a medical worker, for a patient, and for an insurance examination. For example, a prompt text such as “Based on the medical data of this patient, please professionally explain the diagnosis basis for a doctor” or “Please explain the diagnosis basis in an easy-to-understand manner so that the patient can be convinced” can be given to the generative AI. Specifically, the AI diagnosis transparency unit executes a large language model pipeline incorporating a retrieval-augmented generation technology. The AI diagnosis transparency unit receives a latest test numerical vector of the patient, text data of a past medical record, and a categorical variable designating a target readership as input data. The AI diagnosis transparency unit acquires, for example, an array of floating-point numbers including 30 items of blood test results as the input data. The AI diagnosis transparency unit also acquires a flag designating an output style of “for insurance examination” as the input data. The AI diagnosis transparency unit extracts a text chunk of a latest medical guideline or a similar case from a vector database by similarity search as preprocessing. The AI diagnosis transparency unit integrates the extracted external knowledge and the input data of the patient, and dynamically constructs a structured prompt. The AI diagnosis transparency unit inputs this prompt to a transformer model having billions of parameters. The AI diagnosis transparency unit calculates a relevance between an abnormal value of a test numerical value and a description of the medical guideline using a multi-head attention mechanism inside the model. The AI diagnosis transparency unit outputs a list of diagnosis names, a certainty score of each diagnosis, and a text of a basis explanation optimized for the designated readership as output data. The AI diagnosis transparency unit outputs, for example, a score of a certainty of 0.98 for type 2 diabetes as the output data. The AI diagnosis transparency unit also outputs an ICD code necessary for an insurance claim and a standard explanatory text indicating medical validity as the output data. The AI diagnosis transparency unit transmits this output result to a subsequent approval workflow module. When the certainty score satisfies a certain criterion, the approval workflow module executes a branching process of automatically creating an insurance claim draft. In learning of the model, the AI diagnosis transparency unit performs fine-tuning using a chain-of-thought prompting method describing a thinking process of a medical specialist. As a technical effect, the AI diagnosis transparency unit fulfills accountability for an inference result of the AI, strongly supports a final decision-making of a medical worker, and streamlines an administrative procedure such as an insurance claim. As a specific application field, the AI diagnosis transparency unit is utilized in an automated system for receipt inspection or a medical consultation platform providing a second opinion.

The patient explanation generation unit has a function of automatically generating an explanatory text in a natural language easy for a patient to understand using generative AI based on the diagnosis result and the diagnosis basis generated by the AI diagnosis transparency unit. The patient explanation generation unit gives a prompt text such as “Based on this diagnosis result and diagnosis basis, please create an explanatory text that is easy for the patient to be convinced”, “Please explain a treatment policy in an expression easy for an elderly person to understand”, or “Please explain to a guardian of a pediatric patient including future points to note” to the generative AI, and generates an explanatory text considering an age, a sex, a past medical history, an understanding level, a language setting, and the like of the patient. For example, for a patient diagnosed with hypertension, the patient explanation generation unit generates an explanatory text such as “Your blood pressure is 150/95 mmHg, which exceeds the reference value (140/90 mmHg), so you have been diagnosed with hypertension. In the future, salt reduction, exercise, and taking an antihypertensive drug as necessary are recommended.” In addition, when diagnosed with suspected diabetes, the patient explanation generation unit generates an explanatory text such as “There is a possibility of diabetes because the blood glucose level is high, and an additional test and lifestyle guidance are necessary.” Furthermore, the patient explanation generation unit can provide the generated explanatory text by various methods such as displaying on a doctor terminal or a patient terminal (smartphone, tablet, personal computer, etc.), printing, transmitting by e-mail, and posting on a patient portal site. Specifically, the patient explanation generation unit executes a process integrating a style conversion technology and a personalization algorithm in natural language processing. The patient explanation generation unit receives text data of a diagnosis basis including a technical term and a profile vector including demographic information of the patient as input data. The patient explanation generation unit acquires, for example, a technical text “suspicion of left ventricular hypertrophy due to essential hypertension” as the input data. The patient explanation generation unit also acquires a vector including a flag indicating that the age of the patient is 75 years old and the visual acuity is reduced as the input data. The patient explanation generation unit refers to a technical term dictionary and maps a difficult medical term to a plain concept token as preprocessing. The patient explanation generation unit inputs this token sequence and the profile vector to a conditional language model. The patient explanation generation unit dynamically adjusts a selection probability of a vocabulary according to the understanding level of the patient in a decoder layer of the model. The patient explanation generation unit outputs a text of an explanatory text converted into a plain expression and markup data including an SSML tag for speech synthesis as output data. The patient explanation generation unit outputs, for example, a text “Because a state of high blood pressure continues, there is a possibility that a wall of the heart is thickened” as the output data. The patient explanation generation unit also outputs a speech synthesis tag designating a slow reading speed as the output data. The patient explanation generation unit transmits this output result to a subsequent multimodal presentation module. The multimodal presentation module executes a branching process of displaying the text on a screen with a large font or outputting the text from a speaker as a synthesized speech according to a terminal setting of the patient. In learning of the model, the patient explanation generation unit performs learning as a translation task from a technical term to a plain expression using a parallel corpus created by an expert in medical communication. As a technical effect, the patient explanation generation unit bridges a gap in health literacy of patients, and improves compliance with treatment and patient satisfaction. As a specific application field, the patient explanation generation unit is used in an automatic creation system of a medical treatment guidance document at the time of discharge or a multilingual medical interpretation application.

The inter-hospital data linkage unit has a function of encrypting the medical data recorded by the medical data management unit and enabling secure sharing with another medical institution. For example, when a patient transfers to another hospital, the inter-hospital data linkage unit can encrypt a medical history of the patient in a server of a transfer source medical institution and transmit it to a server of a transfer destination medical institution via a secure communication path. In addition, when a patient is receiving treatment at a plurality of medical institutions, servers of the respective medical institutions can mutually encrypt and share medical data, and comprehensively grasp the entire medical history of the patient. For example, at the time of emergency transportation, an emergency hospital can immediately acquire the medical history of the patient, refer to a past medical history, allergy information, and latest prescription drug information, and determine an optimal treatment policy. The inter-hospital data linkage unit executes encryption/decryption processing on the server side, and viewing and inputting of decrypted data are performed on the terminal side. Furthermore, when sharing the medical data, the inter-hospital data linkage unit can give a prompt text such as “In order to share the medical history of this patient with another hospital, please summarize necessary information and output it in a form considering personal information protection” to the generative AI, and automatically perform summarization and anonymization of the shared data. Specifically, the inter-hospital data linkage unit adopts a data federation architecture based on a zero-trust security model. The inter-hospital data linkage unit applies an attribute-based encryption method to data to be shared. The inter-hospital data linkage unit receives JSON data of a medical record to be shared and a policy document defining an attribute condition of a medical worker permitted to access as input data. The inter-hospital data linkage unit acquires, for example, a list of a past prescription history of the patient as the input data. The inter-hospital data linkage unit also acquires a policy that enables decryption only by a user having an attribute of “emergency life-saving technician” or “cardiologist” as the input data. The inter-hospital data linkage unit detects direct personal identification information such as a name and an address of the patient from the text data using a named entity extraction model of the generative AI, and executes a masking process as preprocessing. The inter-hospital data linkage unit converts this anonymized data into a ciphertext tensor using an attribute-based encryption algorithm. The inter-hospital data linkage unit outputs an encrypted data payload and a metadata header in which an access control policy is embedded as output data. The inter-hospital data linkage unit outputs, for example, an encrypted data block in a binary format as the output data. The inter-hospital data linkage unit transmits this output result to a subsequent distributed messaging queue. The messaging queue executes a process of asynchronously distributing the encrypted data in response to a data request from a medical institution on the receiving side. The inter-hospital data linkage unit verifies an attribute certificate of the user on the receiving side, and generates a decryption key to restore the data only when the policy is met. As a technical effect, the inter-hospital data linkage unit enables secure and on-demand sharing of only necessary information while retaining ownership of the data in the original medical institution, and minimizes an information leakage risk. As a specific application field, the inter-hospital data linkage unit provides an indispensable function in a multidisciplinary cooperation platform in a community-based comprehensive care system or a wide-area medical information backup system in the event of a disaster.

The falsification detection unit has a function of automatically detecting presence or absence of falsification by verifying the hash chain of the medical data. For example, when an unauthorized rewriting is performed on the medical history, the falsification detection unit can specify a falsified portion by verifying the hash chain, and notify an administrator or a medical worker of a warning. Furthermore, the falsification detection unit analyzes whether an unnatural change or an abnormal pattern is included in the content of the medical data using generative AI, automatically detects a pattern with a high possibility of falsification or fraud, for example, when a part of a medical record is different from a normal medical practice, when a plurality of medical data are collectively changed in a short period of time, or when there is a contradiction between the medical content and the diagnosis result, and generates and notifies warning information. The falsification detection unit can perform analysis by giving a prompt text such as “Please analyze in detail from a medical viewpoint whether there is an unnatural change or an abnormal pattern in this medical history, and point out a suspicious portion if any” to the generative AI. Specifically, the falsification detection unit executes a cryptographic verification module and a semantic anomaly detection module based on natural language processing in parallel. In the cryptographic verification, the falsification detection unit hierarchically calculates hash values of all records in the database using a Merkle tree data structure, and periodically anchors a root hash to a public blockchain. By this process, the falsification detection unit ensures decentralized provability that even a system administrator cannot falsify past data. In the semantic anomaly detection, the falsification detection unit receives text difference data of the medical record before and after an update and a behavior log vector of a user who executed the update as input data. The falsification detection unit acquires, for example, a difference text indicating that a dose of a prescription drug has been changed from “10 mg” to “100 mg” as the input data. The falsification detection unit also acquires a time stamp vector indicating that an update time is 2:00 a.m. as the input data. The falsification detection unit combines these data and converts them into a token sequence that can be input to a pre-trained language model as preprocessing. The falsification detection unit inputs this sequence to a transformer-based classification model, and evaluates validity of the change. The falsification detection unit outputs an anomaly score from 0 to 1 indicating a degree of suspicion of falsification and a text explaining a reason for being determined as an anomaly as output data. The falsification detection unit outputs, for example, an anomaly score of 0.92 as the output data. The falsification detection unit also outputs a warning text “A change significantly exceeding a normal prescription dose is made in an unusual time zone” as the output data. The falsification detection unit transmits this output result to a subsequent incident response module. When the anomaly score exceeds a threshold value, the incident response module executes a branching process of temporarily freezing an account of a corresponding user and preserving an audit log. As a technical effect, the falsification detection unit multi-layeredly defends against not only simple data corruption but also clever data manipulation by an internal crime, and firmly secures legal evidence capability of the medical data. As a specific application field, the falsification detection unit is used as an integrity assurance system for clinical trial data or an internal audit support tool for a medical institution.

Furthermore, the medical data management unit and the AI diagnosis transparency unit also have a function of automatically generating a summary text of the medical data using generative AI. For example, a prompt text such as “Please summarize the medical history of this patient for the past 10 years, and briefly summarize a main diagnosis and treatment progress” is given to the generative AI to enable a medical worker to grasp an entire picture of the patient in a short time. The medical data management unit can record the summary text as a part of the medical history, and enable viewing on a doctor terminal or a nurse terminal. Specifically, the system utilizes a large language model having a sparse attention mechanism optimized for processing a long-text context. The system receives text data of hundreds of medical records over the past several years, chronological numerical data of test results, and a template designating a format of a summary as input data. The system acquires, for example, a text sequence of tens of thousands of tokens in which medical chart records in a SOAP format for each consultation date are connected in chronological order as the input data. The system also acquires a template requesting items of “history of present illness”, “past medical history”, and “current prescription” as the input data. The system divides the text sequence into chunks as preprocessing, evaluates an importance of each chunk in advance, and filters information that becomes noise. The system inputs the filtered important chunks to the language model, and extracts and integrates information according to the designated template. The system outputs a structured summary text and an array of reference links indicating which original medical record each summary item is based on as output data. The system outputs, for example, a summary text of about 500 characters organized in bullet points as the output data. The system also outputs a URI list of an original medical chart linked to a specific disease name in the summary text as the output data. The system transmits this output result to a subsequent database update module. The database update module executes a process of storing the generated summary text as a cache in a master record of the patient and displaying it at high speed at the next data reading. In learning of the model, the system performs fine-tuning reflecting human feedback by reinforcement learning using a high-quality summary dataset created by a medical specialist. As a technical effect, the system dramatically reduces a cognitive load and time for a medical worker to grasp a medical history of a patient, and realizes higher-quality medical communication within a limited consultation time. As a specific application field, the system functions extremely effectively as an instantaneous grasping system of patient information in an emergency outpatient department or a preliminary information organizing tool for a referred patient from another hospital.

As described above, the system according to the present embodiment comprises the medical data management unit, the AI diagnosis transparency unit, the patient explanation generation unit, the inter-hospital data linkage unit, and the falsification detection unit, and each unit utilizes generative AI to integrally and specifically realize various functions such as recording, management, diagnosis, explanation, sharing, falsification detection, and summarization of medical data, thereby dramatically improving transparency, reliability, explainability, safety, and efficiency of medical data in a medical field. Specifically, the system forms an autonomous ecosystem in which each functional module not only operates independently but also cooperates with each other via an event-driven architecture. The system adopts an asynchronous communication infrastructure centered on a message broker, and a data update in a certain module triggers processing of another module in real time. For example, the system detects an event in which a new test result is registered in the medical data management unit, and automatically executes a chain of re-evaluation by the AI diagnosis transparency unit and update of an explanatory text by the patient explanation generation unit. The system monitors a data flow of the entire system, quantifies uncertainty of an inference result of each AI model, and visualizes it on a dashboard. As a technical effect, the system highly automates an end-to-end process from input to utilization of medical data, and secures quality and safety of a medical service at a system level while eliminating room for intervention of a human error. As a specific application field, the system plays a role of driving medical digital transformation as a foundation system of an integrated healthcare platform in a smart city or a core medical information system in a next-generation advanced acute care hospital.

### Implementation Steps

### Step 1: Collection and Recording of Medical Data

A medical worker inputs patient identification information, medical content, vital data, a test result, a diagnosis result, a diagnosis basis, a recording time, and the like using an outpatient reception terminal, a tablet terminal for nurses, a test equipment linkage system, or the like, and transmits them to the medical data management unit on the server. The medical data management unit records the received data for each patient in chronological order, assigns a hash value of immediately preceding medical data to each medical data, and stores the medical data as a hash chain. For example, when a patient undergoes a blood test in the morning and a medical examination by a doctor in the afternoon, each medical data is independently recorded, and a hash value of the medical data of the morning is assigned to the medical data of the afternoon. Specifically, in this step, the system executes a secure data ingest pipeline between an edge device and a cloud server. The system receives a structured data payload in an HL7 FHIR format output from various terminals and test equipment as input data. The system acquires, for example, a binary array of waveform data output from an electrocardiograph and a metadata JSON including a patient ID and a measurement time accompanying the binary array as the input data. The system performs schema verification of the received data payload as preprocessing, and executes complementation of a missing value and normalization of a unit. The system temporarily buffers the normalized data in an in-memory database, and processes writing with high throughput. When asynchronously writing the buffered data to a persistent storage, the system calculates a hash value of an immediately preceding record using a SHA3 algorithm, and combines it with a header of a current record. The system generates an immutable data block in which a hash chain is formed as output data, and commits it to a distributed database. The system outputs, for example, a data block to which a unique transaction ID is assigned as the output data. The system transmits this output result to a subsequent event publisher. The event publisher issues a registration completion event of new data to a message queue, and executes a branching process of triggering a subsequent AI diagnosis step. As a technical effect, the system reliably collects a large amount of data asynchronously generated from a wide variety of medical devices without omission, and constructs an unfalsifiable chronological data lake. As a specific application field, the system is used in a data collection system of an intensive care unit in which a large number of IoT medical devices operate or a continuous biological data logging system from a wearable device.

### Step 2: Diagnosis and Basis Explanation Generation by AI

The AI diagnosis transparency unit performs a diagnosis using generative AI based on the medical data of the patient acquired from the medical data management unit on the server, and generates the diagnosis basis as an explanatory text. For example, blood test data (blood glucose level, HbA1c, cholesterol level, etc.) of the patient is received as an input, a prompt text such as “Please explain in detail medically possible diseases and their bases based on the blood test data of this patient” is given to the generative AI, and the diagnosis result and the basis explanation are generated. The generative AI also refers to specific data items and medical diagnostic criteria used for the diagnosis, past medical history, family history, lifestyle information, and the like, and outputs a multifaceted diagnosis basis as an explanatory text. Specifically, in this step, the system executes a deep learning pipeline that integrally analyzes multimodal medical data. The system receives, as input data, the latest test numerical vector recorded in Step 1, a text embedding vector of past medical records, and a pixel tensor of a medical image. The system acquires, for example, a one-dimensional tensor obtained by normalizing numerical values of 50 items of a blood test as the input data. The system also acquires a two-dimensional tensor of 256×256 pixels of a chest X-ray image as the input data. As preprocessing, the system inputs these heterogeneous data into a cross-attention network, and generates an integrated feature vector in which a correlation between respective modalities is learned. The system projects this integrated feature vector into a prompt space of a large language model, and combines it with an instruction prompt specialized for a diagnosis task. In the inference process of the model, the system executes inference a plurality of times using a Monte Carlo dropout method to quantify the uncertainty of prediction. The system outputs, as output data, a predicted probability distribution of a disease, an uncertainty score, and an attention map indicating a contribution degree of a feature amount serving as the diagnosis basis. The system outputs, for example, a predicted probability of pneumonia of 0.88 and an uncertainty score of 0.05 as the output data. The system also outputs, as the output data, heat map data indicating an abnormal shadow region on the X-ray image and a medical explanatory text corresponding thereto. The system transmits this output result to a subsequent patient explanation generation step. The system executes a branching process of setting a flag requesting a review by a human medical specialist when the predicted probability falls below a threshold value or the uncertainty score exceeds a threshold value. As a technical effect, the system realizes comprehensive diagnostic inference that does not depend on a single data source, and quantitatively and visually presents the judgment basis of the AI, thereby increasing the acceptability of the AI in the medical field. As specific application fields, the system is utilized as a differential diagnosis support system for complex cases in a general practice department and a primary screening tool in a remote image diagnosis service.

### Step 3: Automatic Generation of Explanatory Text for Patient

The patient explanation generation unit automatically generates an explanatory text in a natural language easy for a patient to understand using generative AI based on the diagnosis result and the diagnosis basis received from the AI diagnosis transparency unit. For example, a prompt text such as “Please create an explanatory text that is easy for the patient to understand based on this diagnosis result and diagnosis basis,” “Please explain the treatment policy in an expression easy for an elderly person to understand,” or “Please explain to a guardian of a pediatric patient including future points to note” is given to the generative AI, and an explanatory text considering the age, sex, medical history, comprehension level, language setting, and the like of the patient is generated. Specifically, in this step, the system dynamically generates personalized medical communication content using a text generation model optimized by reinforcement learning. The system receives, as input data, structured data of the diagnosis result generated in Step 2, a medical text serving as a basis, and a demographic attribute vector extracted from an electronic medical record of the patient. The system acquires, for example, a code list of a disease name, severity, and a recommended treatment method as the input data. The system also acquires, as the input data, a 128-dimensional attribute vector encoding the mother tongue, education level, and past medical compliance history of the patient. As preprocessing, the system maps these input data to a prompt template, and constructs a token sequence that can be input to a context window of a language model. In a decoder network, the system uses the attribute vector of the patient as a conditioning signal to dynamically bias a probability distribution of output words. The system outputs, as output data, a plain natural language text tailored to the comprehension level of the patient, and visual format data for emphasizing important information. The system outputs, for example, a text string of an explanatory text excluding technical terms and using a metaphor as the output data. The system also outputs, as the output data, JSON data for generating an infographic for illustrating a medication schedule. The system transmits this output result to a subsequent distribution module. The distribution module executes a branching process of rendering the content in an optimal layout according to the terminal type (smartphone, tablet, printed matter, etc.) of the patient. As a technical effect, the system automates tailor-made medical information provision tailored to the background of each patient, improves the quality of informed consent, and significantly reduces the time required for explanation work by a medical worker. As specific application fields, the system is used as automatic creation of a personalized medical treatment plan in a discharge support system and a personal health assistant function in a portal site for patients.

### Step 4: Summary Generation of Medical Data

The medical data management unit and the AI diagnosis transparency unit automatically generate a summary text of the entire medical history of the patient using generative AI. For example, a prompt text such as “Please summarize the medical history of this patient over the past 10 years and briefly summarize the main diagnosis and treatment progress” is given to the generative AI to enable a medical worker to grasp the entire picture of the patient in a short time. The summary text is recorded as a part of the medical history and can be viewed on a doctor terminal or a nurse terminal. Specifically, the medical data management unit of the system extracts all past medical records of the target patient from a distributed database. The processing unit inputs the extracted text data and numerical data to a natural language processing module. The natural language processing module utilizes a large language model equipped with a pre-trained self-attention mechanism. The model converts the input time-series medical data into a high-dimensional word embedding vector. The input data to the model is, for example, time-series array data including transitions of consultation dates, diagnosis names, prescription drugs, and test values over the past 10 years. The number of dimensions of an input tensor to the model is configured as, for example, a third-order tensor consisting of a batch size, a sequence length, and the number of dimensions of a hidden layer. The model executes a multi-head attention operation on the input tensor. By this operation, the model extracts a contextual dependency relationship between medical records over a long period of time. The model inputs the extracted feature vector to a decoder network. The decoder network autoregressively outputs a token probability distribution of a summary text. The output data from the model is a probability distribution vector of a vocabulary size at each time step. The model generates an optimized summary text character string using a beam search algorithm. Specific examples of the output of the model include structured summary texts such as “Diagnosed with type 2 diabetes in 2015 and started metformin administration,” “Transitioned to insulin therapy in 2020 because HbA1c worsened to 8.5 percent,” and “Blood glucose control is good in the latest test.” The system transfers the generated summary text to a subsequent display processing module. The display processing module renders the summary text in a predetermined area of a dashboard screen of an electronic medical record system. By this processing, the system reduces the time for a medical worker to view a huge amount of past medical records one by one. The system reduces the consumption of computer resources required for information retrieval. By automating the summary generation processing, the system brings about a technical effect of speeding up data grasping in the medical field. This technology is extremely useful in specific use cases such as rapid grasping of a patient background during emergency transport and medical interview support at the time of an initial consultation. In the learning process of the model, the system uses a pair of a discharge summary created by a doctor in the past and an original medical record as training data. The system calculates a cross-entropy error between the generated summary and the training data. The system optimizes a weight parameter of the model using an error backpropagation method. By this learning method, the system realizes highly accurate summary generation specialized for the medical domain.

### Step 5: Inter-Hospital Data Linkage and Secure Sharing

The inter-hospital data linkage unit encrypts the medical data recorded by the medical data management unit, and securely shares it with another medical institution. For example, when a patient transfers to another hospital, a server of a transfer source medical institution encrypts a medical history of the patient, and transmits it to a server of a transfer destination medical institution via a secure communication path. In the transfer destination medical institution, the server side can decrypt the medical history of the patient and integrate it into its own electronic medical record system. Furthermore, when sharing the medical data, a prompt text such as “Please summarize necessary information and output it in a form considering personal information protection in order to share the medical history of this patient with another hospital” is given to the generative AI, and summarization and anonymization of the shared data are also automatically performed. Specifically, the inter-hospital data linkage unit of the system extracts medical data to be shared. The linkage unit applies an advanced encryption standard algorithm to the extracted data. The linkage unit encrypts the data in block units using, for example, a 256-bit common key. The linkage unit encapsulates the common key using a public key cryptography method. The linkage unit combines the encrypted data payload and the encapsulated common key to generate a transmission packet. The linkage unit transmits the generated transmission packet to the server of the transfer destination via a virtual private network. The system executes data summarization and anonymization processing using generative AI before transmission. The system masks direct personal identification information such as the name and address of the patient. The system inputs the masked text data to a large language model. The input data to the model includes, for example, a masked medical progress text, a numerical vector of test values, and a prompt character string specifying a sharing purpose. The model extracts medical information essential for sharing from the input data. The output data from the model is structured text data for which anonymization and summarization have been completed. Specific examples of the output of the model include information such as “Patient A underwent stent placement for acute myocardial infarction in 2022,” “Currently taking an antiplatelet drug,” and “Has a rash to penicillin as an allergy history.” The system inputs the output structured text data to a subsequent encryption module. By this processing, the system realizes secure transfer of necessary and sufficient medical information while complying with personal information protection legislation. The system exhibits a technical effect of suppressing consumption of a communication band by reducing a data transfer amount. This technology is applicable to use cases such as inter-facility data sharing in a regional medical linkage network and backup data recovery in the event of a disaster.

### Step 6: Falsification Detection and Abnormality Analysis of Medical Data

The falsification detection unit verifies the hash chain of the medical data on the server, and automatically detects the presence or absence of falsification. Furthermore, the falsification detection unit analyzes whether an unnatural change or an abnormal pattern is included in the content of the medical data using generative AI. For example, a prompt text such as “Please analyze in detail from a medical viewpoint whether there is an unnatural change or an abnormal pattern in this medical history, and point out any suspicious part” is given to the generative AI to perform analysis, a pattern with a high possibility of falsification or fraud is automatically detected, and warning information is generated and notified. Specifically, the falsification detection unit of the system sequentially reads the hash chain of the medical data stored in the database. The detection unit combines the data content of each record and the hash value of the immediately preceding record. The detection unit applies a secure hash algorithm to the combined data to calculate a new hash value. The detection unit compares the calculated hash value with the recorded hash value. When the two do not match, the detection unit determines that the data after the corresponding record has been falsified. In addition to the hash chain verification, the system executes semantic abnormality detection using generative AI. The system inputs a time-series medical record text and a test value vector to a large language model. The input data to the model includes, for example, a time-series array of a prescription history for the past year, text data of a chief complaint on each consultation date, and an identifier of an attending doctor. The model maps the data to a latent space in which a standard transition pattern of medical practice is learned from the input time-series data. The model calculates a degree of divergence between the input data and the standard pattern as an abnormality score. The output data from the model is an abnormality score vector taking a continuous value from 0 to 1 for each medical record. Specific examples of the output of the model include a score of 0.95 for a record in which an expensive self-pay medical treatment is suddenly added on a specific day, and a score of 0.88 for a record in which a strong analgesic completely unrelated to a past diagnosis name is prescribed. The system determines whether the output abnormality score exceeds a preset threshold value. When the threshold value is exceeded, the system activates a subsequent warning generation module. The warning generation module transmits an immediate alert to a terminal of a system administrator. The system realizes not only simple cryptographic verification but also advanced fraud detection based on a medical context. The system brings about a technical effect of early discovering clever data falsification by an internal crime. This technology is extremely effective in use cases such as evidence preservation in medical lawsuits and prevention of fraudulent billing of medical insurance.

### Step 7: Terminal Display and Utilization of Explanatory Text and Medical History

The explanatory text for a patient, the medical data summary, the AI diagnosis result, and the like generated on the server side are provided by various methods such as display, printing, e-mail transmission, and posting on a patient portal site on a doctor terminal or a patient terminal (smartphone, tablet, personal computer, etc.) so that a medical worker or a patient can easily grasp and utilize the medical content and the treatment policy. Specifically, the display processing unit of the system receives various text data and analysis results generated on the server side. The display processing unit converts the received data into a display markup language format optimized for the screen resolution and operating system of the terminal. The display processing unit transmits the converted data to each terminal via a hypertext transfer protocol. For the doctor terminal, the system superimposes and displays a detailed diagnosis basis including technical medical terms and a time-series test value graph. For the patient terminal, the system provides an explanatory text in which technical terms are replaced with plain expressions, and a user interface using illustrations and icons that are visually easy to understand. When generating the display data, the system dynamically adjusts the text according to the health literacy level of the patient using generative AI. The input data to the AI is, for example, vector data of a standard explanatory text for a patient, the age, final educational background, and past medical information browsing history of the patient. The AI adjusts the difficulty level of the text based on the input data. The output data from the AI is a customized explanatory text optimized for each patient. Specific examples of the output of the AI include a text in which the character size is increased and “Please take your medicine at a fixed time every day” is emphasized for elderly people, and a text including detailed numerical data and a specific action plan for lifestyle improvement for young people. The system inputs the output text to a subsequent rendering engine, and draws it on the screen. The system completes heavy drawing processing and text generation processing on the server side without depending on the hardware resources of the terminal. The system exhibits a technical effect of significantly reducing battery consumption and processing load on the terminal side. This technology is applied to use cases such as real-time information provision to a patient in remote medical care and a self-management support application after discharge.

### Specific Use Case

For example, assume a case where a patient visits a plurality of medical institutions and continues treatment for chronic hypertension and diabetes. In this case, the medical data management unit records a blood pressure value, a blood glucose level, a body weight, medication content, a diagnosis result by a doctor, lifestyle guidance content, a test result, and the like measured when the patient has an outpatient consultation in chronological order. Furthermore, the content of tests and treatments received by the patient at another hospital is also securely received in an encrypted state via the inter-hospital data linkage unit, and is integrally managed as a unified medical history of the patient. For example, when the patient undergoes a blood test at a medical institution A in the morning and has a consultation at a medical institution B in the afternoon, the medical data management unit records the respective medical data independently, assigns the hash value of the morning medical data to the afternoon medical data, and stores it as a hash chain. As a result, even when there is falsification or unauthorized rewriting of the medical data, the falsification detection unit can verify the consistency of the hash chain and immediately detect an abnormality. Specifically, the medical data management unit of the system receives medical data streams asynchronously transmitted from a plurality of medical institutions. The management unit extracts an identifier of the patient from the received data stream. The management unit specifies a patient profile on a distributed database using the extracted identifier. The management unit expands the received numerical data such as the blood pressure value, the blood glucose level, and the body weight on a memory as an array of floating-point numbers. The management unit acquires text data such as the medication content and the findings of the doctor as a character string. The system normalizes these heterogeneous mixed data according to a unified data schema. The system assigns a time stamp and a medical institution identifier of a data generation source to the normalized data record. The system calculates a cryptographic hash value of the immediately preceding recorded data record. The system embeds the calculated hash value in a header portion of the current data record. By repeating this processing, the system constructs a hash chain structure in which a temporal order is guaranteed. The system persists the constructed hash chain in a non-volatile storage. The system periodically activates the falsification detection unit. The detection unit reads the hash chain from the storage, recalculates the hash value of each block, and verifies the consistency. With this data structure, the system mathematically guarantees the integrity and traceability of data even in an environment where writing occurs from a plurality of nodes. The system brings about a technical effect of realizing high-throughput data recording while avoiding lock contention of the database. This technology plays a core role in use cases such as a multidisciplinary linkage platform in a community-based integrated care system and a lifelong personal health record management system.

The AI diagnosis transparency unit instructs the generative AI to perform a diagnosis and a basis explanation using the latest blood pressure value, blood glucose level, HbA1c, medication status, medical history, family history, lifestyle information, and the like of the patient acquired from the medical data management unit as input data. For example, a prompt text such as “Please explain in detail medically possible diseases and their bases based on the blood pressure value, blood glucose level, HbA1c, medication status, medical history, family history, and lifestyle information of this patient” is given to the generative AI. Based on the fact that the blood pressure value exceeds a reference value, the blood glucose level and HbA1c are high, the high values continue in the past medical history, there is a diabetic patient in the family, and lack of exercise and a high-salt diet are recognized as lifestyle habits, the generative AI outputs a diagnosis basis explanation such as “Poor control of hypertension and diabetes is suspected. The blood pressure is 150/95 mmHg, the blood glucose level is 220 mg/dL, and HbA1c is 8.0%, and the high values continue in the past three tests, suggesting chronic poor control. Family history and lifestyle are also risk factors.” Specifically, the AI diagnosis transparency unit of the system inputs multidimensional patient data extracted from the medical data management unit to a preprocessing module. The preprocessing module normalizes continuous values such as the blood pressure value and the blood glucose level, and converts categorical variables such as the medical history and the family history into a one-hot vector. The system combines the preprocessed data to generate a single input feature vector. The system inputs this feature vector and a prompt character string including a diagnosis instruction to a large language model. The input data to the model includes, for example, numerical data of a systolic blood pressure of 150, a diastolic blood pressure of 95, a blood glucose level of 220, and HbA1c of 8.0, and text data of lifestyle habits of “lack of exercise” and “high-salt diet”. The number of dimensions of an input tensor of the model is represented by, for example, a product of a sequence length of a tokenized text and an embedding dimension. The model applies a multi-layer transformer block to the input tensor, and executes inference while collating with a medical knowledge base. The output data from the model is a probability distribution vector of a disease and a natural language text explaining the basis thereof. Specific examples of the output of the model include score data of “probability of hypertension 95 percent, probability of type 2diabetes 92 percent” and a basis text of “the blood glucose level continuously exceeds 200 in the past three tests, suggesting chronic poor control.” The system selects a disease whose output probability score exceeds a predetermined threshold value as a final diagnosis candidate. The system transfers the selected diagnosis candidate and the basis text to the subsequent patient explanation generation unit. The system verbalizes the inference process of the AI, which tends to be a black box, based on specific numerical values and medical bases. The system exhibits a technical effect of supporting a diagnosis work of a doctor and reducing diagnosis errors. This technology is applicable to use cases such as diagnosis support in remote medical care where a medical specialist is absent and an educational tool for young doctors. In the learning of the model, the system executes fine-tuning using a pair of a diagnosis record of a medical specialist in the past and a basis explanation as training data.

The patient explanation generation unit gives a prompt text such as “Please create an explanatory text that is easy for the patient to understand based on this diagnosis result and diagnosis basis. Please explain including a treatment policy and lifestyle guidance in an expression easy for an elderly person to understand” to the generative AI based on the diagnosis result and the diagnosis basis generated by the AI diagnosis transparency unit. The generative AI automatically generates an explanatory text for a patient such as “Your blood pressure and blood glucose level continue to be high. Your blood pressure is 150/95 mmHg and your blood glucose level is 220 mg/dL, and similar values continue in past tests. If these conditions continue, a burden will be placed on your heart and kidneys, so please keep the salt content of your meals moderate and try to exercise for about 30 minutes every day. We will also adjust your medicine as necessary, so please follow the instructions of your doctor.” Specifically, the patient explanation generation unit of the system receives the specialized diagnosis result and the basis text output from the AI diagnosis transparency unit. The generation unit combines the received text data and a context vector including attribute information of the patient. The generation unit inputs the combined data to a large language model specialized for natural language generation. The input data to the model includes, for example, structured data of “diagnosis name: hypertension, basis: blood pressure 150/95 continues for 3 months” and control parameters of “age: 75 years old, comprehension level: general, tone: friendly”. The model maps technical terms to a general vocabulary according to the input control parameters. The model generates a plain explanatory text for a patient through an autoregressive decoding process. The output data from the model is character string data of a natural language optimized for the health literacy of the patient. Specific examples of the output of the model include texts prompting specific behavioral changes such as “Days with slightly high blood pressure continue,” “If this continues, it will put a strain on your heart, so let's leave the soup of miso soup,” and “It is good to make time to walk even a little every day.” The system outputs the generated text data to a subsequent voice synthesis module or display rendering module. The system transmits the output text as a push notification on a smartphone application of the patient. The system eliminates the mental and temporal burden of a medical worker devising and creating an explanatory text tailored to each patient. The system brings about a technical effect of equalizing the quality of medical communication and improving the treatment adherence of the patient through automation by a computer. This technology is extremely effective in use cases such as automatic transmission of a follow-up message after online medical care and digitalization of medication guidance.

The medical data management unit and the AI diagnosis transparency unit give a prompt text such as “Please summarize the medical history of this patient over the past five years and briefly summarize the main diagnosis and treatment progress” to the generative AI, and generate a summary text such as, for example, “Continued hypertension treatment over the past five years, diagnosed with diabetes two years ago, started insulin treatment one year ago, and recently poor control of blood pressure and blood glucose level continues”. The summary text is recorded as a part of the medical history and can be viewed on a doctor terminal or a nurse terminal. Specifically, the medical data management unit of the present system extracts all medical records of the target patient over the past five years from a distributed storage by a query search. The management unit sorts the extracted record group in chronological order to construct a single huge text corpus. The present system divides this text corpus into chunk units to adapt to an input limit size of a large language model. The present system inputs each divided chunk to the large language model to generate an intermediate summary vector. The input data to the model is, for example, time-series data including a chief complaint on each consultation day, a numerical array of test results, and a list of prescribed drugs. The model drives an attention mechanism that extracts medically important events from the input data. The output data from the model is a summary text of the important events extracted from each chunk, and a final overall summary text integrating them. Specific examples of the output of the model include a chronological bulleted text such as “2018: Diagnosis of hypertension and start of amlodipine administration”, “2021: Diagnosis of diabetes due to elevated HbA1c”, and “2022: Introduction of insulin therapy”, and a summary text such as “There is a complication of hypertension and diabetes, and the most recent control is poor”. The present system stores the generated summary text as metadata linked to the original medical history database. The present system allows a subsequent search engine to index this summary text. The present system compresses a huge amount of medical data over a long period of time in a high-dimensional space and converts it into a format that can be instantly recognized by a human. The present system dramatically shortens the reading time of an electronic medical record and exhibits a technical effect of improving the efficiency of information processing. The present technology is applied to use cases such as rapid triage of a first-visit patient in an emergency outpatient department and efficiency improvement of handover work when an attending physician is changed.

When a patient transfers to another hospital, the inter-hospital data linkage unit encrypts the medical history of the patient on the server of the transfer source medical institution and transmits it to the server of the transfer destination medical institution via a secure communication path. In the transfer destination medical institution, the server side can decrypt the medical history of the patient and integrate it into its own electronic medical record system. When sharing the medical data, a prompt text such as “In order to share the medical history of this patient with another hospital, please summarize necessary information and output it in a form that considers personal information protection” is given to the generative AI, and the summary and anonymization of the shared data are also automatically performed. Specifically, the inter-hospital data linkage unit of the present system receives a trigger signal for a hospital transfer procedure. The linkage unit extracts all medical data of the target patient from the database. The linkage unit executes a summary and anonymization process using the generative AI on the extracted data. The input data to the AI is, for example, the entire medical history text of the patient, clinical department information of the transfer destination, and a parameter specifying a personal information protection level. The AI preferentially extracts information related to the clinical department of the transfer destination from the input data, and deletes or pseudonymizes direct identifiers such as a name and contact information. The output data from the AI is structured summary text data including only information necessary and sufficient for continuing treatment at the transfer destination. Specific examples of the output of the AI include an anonymized data set such as “Patient ID: XYZ123”, “Main disease name: Colorectal cancer”, “Surgery history: Laparoscopic surgery performed in 2023”, and “Current prescription: Anticancer drug A”. The present system inputs the output summary data to a subsequent encryption module. The encryption module encrypts the data using an elliptic curve cryptography algorithm to generate a secure payload. The present system transfers the generated payload to the server of the transfer destination via a mutually authenticated transport layer security communication path. The server of the transfer destination decrypts the payload using its own private key, and maps the data according to a database schema of its own electronic medical record system. The present system mathematically eliminates a data leakage risk on a communication path while ensuring data interoperability between systems of different medical institutions. The present system brings about a technical effect of achieving both safety and efficiency of data transfer. The present technology becomes an indispensable basic technology in use cases such as seamless sharing of patient information in a wide-area medical network and multi-center joint data collection for clinical research.

The falsification detection unit verifies the hash chain of the medical data and automatically detects the presence or absence of falsification. Furthermore, a prompt text such as “Please analyze in detail from a medical viewpoint whether there is an unnatural change or an abnormal pattern in this medical history, and point out any suspicious part” is given to the generative AI, and a pattern with a high possibility of falsification or fraud, such as a case where a part of the medical record is different from a normal medical practice, a case where a plurality of medical data are changed at once in a short period of time, or a case where there is a contradiction between the medical content and the diagnosis result, is automatically detected, and warning information is generated and notified. Specifically, the falsification detection unit of the present system constantly operates as a background process. Every time a new record is added to the database, the detection unit combines the hash value of the immediately preceding record and the content of the new record to calculate a new hash value. The detection unit cryptographically verifies whether the calculated hash value maintains the consistency of the entire chain. In addition to this deterministic verification, the present system executes probabilistic anomaly detection using the generative AI in parallel. The present system inputs text data and metadata (recorder ID, recording time, etc.) of the target medical record to the large language model. The input data to the model is, for example, a sequence of medical records for the past one month, and a difference vector of timestamps of each record. The model constructs a standard workflow model of medical practice from the input data, and calculates how much the input data deviates from the model. The output data from the model is an anomaly probability score for each record, and text data indicating a reason determined to be abnormal. Specific examples of the output of the model include analysis results such as “Score 0.92 because the recording time is 3:00 midnight, which is outside normal consultation hours” and “Score 0.89 because expensive tests contradicting past diagnosis names are continuously added”. When the output anomaly probability score exceeds a set threshold value, the present system activates a subsequent alert issuing module. The module highlights an identifier of the suspicious record and an analysis reason on a dashboard of an administrator. The present system contextually discovers clever data manipulation abusing medical knowledge, which is difficult to detect by a simple rule base. The present system exhibits a technical effect of dramatically improving the reliability of medical data. The present technology is applicable to use cases such as automation of external audits for medical institutions and security monitoring of electronic medical record systems.

As described above, the system of the present invention can highly and efficiently realize unified management of medical data at multiple facilities and multiple time points, transparency of a diagnosis basis by AI, automatic generation of an explanatory text for a patient, summary of a medical history, secure data linkage between hospitals, falsification detection of medical data, and the like using generative AI. Specifically, the present system adopts an architecture in which a plurality of microservice groups constructed on a distributed computing environment operate cooperatively. The present system deploys each processing unit as an independent container, and dynamically routes a request via a load balancer. The present system seamlessly links construction of an immutable data store using a hash chain by the medical data management unit, integration of inference in a high-dimensional vector space and natural language generation by the AI diagnosis transparency unit, dynamic text rendering according to a user context by the patient explanation generation unit, secure data routing using a public key infrastructure by the inter-hospital data linkage unit, and hybrid processing of cryptographic verification and semantic anomaly detection by the falsification detection unit. In these processes, the present system ensures real-time response performance by offloading an inference pipeline of a large language model to a parallel computing core of a graphics processing unit. The present system dramatically reduces a work load of a medical worker by entrusting a computer with processing of a huge amount of medical data exceeding the limit of human cognitive ability. The present system brings about an improvement in computer technology itself, which enables data-driven, highly accurate medical provision, not limited to mere computerization of business. The present system exhibits complex technical effects of preventing medical malpractice, respecting a patient's right to self-determination, and optimally allocating medical resources. The present technology is an extremely innovative solution that demonstrates its true value in large-scale use cases such as a basic system of a next-generation smart hospital concept and construction of a national-level medical information platform.

(Application Example 1) The flow of the specific processing in Application Example 1 will be described. Each part of the system described below is realized by the data processing device 12 and the smart device 14. Additionally, the data processing device 12 is referred to as a “server,” and the smart device 14 is referred to as a “terminal.”

### Embodiment for Carrying out the Present Invention

Embodiments of the present invention will be described more specifically and in detail. The system of the present invention comprises a nursing care record management unit, a care plan AI determination unit, an explanation generation unit, a data linkage unit, and a falsification detection unit, and these units cooperate to highly support all operations in a nursing care site. Specifically, the present system adopts a scalable distributed processing architecture constructed on a cloud computing environment. The present system implements each functional unit as an independent microservice, and dynamically allocates resources using a container orchestration tool. The present system collects data in real time via an application programming interface from a large number of sensor devices installed in a nursing care facility and mobile terminals of staff. The present system asynchronously distributes the collected data to each processing unit via a message queuing system. The present system operates a hybrid inference engine combining a deep learning-based large language model and a graph neural network in the care plan AI determination unit. The present system dynamically generates a text tailored to a persona of each user using natural language processing technology in the explanation generation unit. The present system implements an end-to-end encrypted communication protocol in the data linkage unit to guarantee secure data exchange with an external system. The present system applies a distributed ledger algorithm similar to blockchain technology in the falsification detection unit to mathematically prove non-falsification of data. By integrating these advanced information processing technologies, the present system breaks through the limits of conventional nursing care record management using paper media or a simple database. The present system promotes digital transformation of nursing care operations, and brings about a technical effect of simultaneously realizing labor saving in data input and sophistication of data utilization. The present technology is applied as an integrated platform that comprehensively manages various forms of nursing care service offices such as a special nursing home for the elderly, a day service center, and a home-visit nursing care office.

First, the nursing care record management unit records and manages nursing care record data for each user in chronological order. For example, when meal assistance is provided at breakfast, staff inputs details such as “meal assistance provided”, “intake 80%”, “no choking”, and “post-meal medication support” from a tablet terminal. When rehabilitation is performed in the morning, rehabilitation content such as “walking training 20 minutes”, “walking distance 50 m”, “no fall”, and “mild fatigue” is recorded. When bathing assistance is provided in the afternoon, information such as “bathing assistance provided”, “no skin condition abnormality”, “watching over when entering and exiting bathtub”, and “low fall risk” is recorded. At night, content of excretion assistance and night watching over, such as “excretion assistance twice”, “night awakening once”, and “good sleep”, is recorded. These records are assigned user identification information, care content, vital data (blood pressure, body temperature, pulse, blood oxygen saturation), a cognitive function score (score such as MMSE or HDS-R), an ADL score (Barthel Index, FIM, etc.), a fall history, a recording time, and a hash value of an immediately preceding record, and are stored in a database on a server. For example, when a user uses a plurality of offices, data recorded in each office is also integrated and managed, and a unified history for each user is constructed. Furthermore, accident/near-miss reports, medication errors, requests and complaints from family members, medical treatments (insulin injection, bedsore treatment, intravenous drip management, etc.), recreation participation status, outing/overnight stay records, emergency transport records, and the like are also recorded in detail. Specifically, the nursing care record management unit of the present system asynchronously receives a wide variety of nursing care record data transmitted from terminals of staff. The management unit classifies the received data into structured data and unstructured data. The management unit expands numerical values such as vital signs and various scores on a memory as an array of floating-point numbers. The management unit acquires text data such as details of care content and special notes as a character string. The present system integrates these data and inserts them into a time-series database for each user. When generating a data record, the present system calculates a cryptographic hash value of an immediately preceding recorded record and assigns it to a header of a current record. By this processing, the present system connects all nursing care records as a hash chain to guarantee the order and completeness of data. The present system also merges data transmitted from a plurality of offices onto a single logical timeline using a user identifier as a key. The present system configures vital data such as blood pressure, body temperature, and pulse as time-series tensor data, for example. The present system normalizes this tensor data into a format usable as an input for a subsequent anomaly detection algorithm or care plan generation model. The present system dynamically reconstructs an index of the database to enable a high-speed search query for past records. The present system realizes instantaneous integration and search of multidimensional data, which is impossible with a paper record book or a distributed file system. The present system exhibits a technical effect of dramatically increasing reliability as an audit trail by making it physically difficult to rewrite or delete data. The present technology plays an extremely important role in use cases such as comprehensive state grasping of a user who uses a plurality of nursing care services together and rapid cause investigation when a nursing care accident occurs.

The care plan AI determination unit automatically determines an optimal care plan by analyzing the latest data and past history data of the user acquired from the nursing care record management unit using generative AI. For example, when the ADL score is low and the cognitive function score is also low, a complex care policy of “physical assistance enhancement+dementia care enhancement” is presented, and a basis explanation such as “Since the ADL score is 20 points or less and almost full assistance is required, and the cognitive function score is 10 points or less and severe dementia is suspected, a two-caregiver system is recommended for all of meals, excretion, bathing, changing clothes, and transfer, and orientation training, environmental adjustment, and wandering countermeasures are enhanced as dementia care” is generated. When there are multiple fall histories, “fall prevention enhancement+environmental improvement+walking training enhancement” is set, and an explanation such as “Since there have been two falls in the past three months and many falls occur when moving to a toilet at night, installation of night lighting, securing a flow line to the toilet, introduction of a walking aid, and enhancement of a night watching over system are recommended” is output. When medication errors occur frequently, “medication management support enhancement” is set, and a basis such as “Since medication errors occur twice or more a week, introduction of a medication support device, calling out at the time of medication, and utilization of a medication checklist are recommended” is shown. When rehabilitation progress is stagnant, “rehabilitation program review” is set, and an explanation such as “Since the walking distance has not increased in the past one month, re-evaluation of rehabilitation content and setting of a new goal are recommended” is generated. Furthermore, referring to the living environment, family structure, medical history, presence or absence of medical treatment, nursing care insurance service utilization status, and the like of the user, highly individualized care plans and bases are output from multiple angles, such as “Since the user is an elderly person living alone and watching over is insufficient, introduction of an emergency call device is recommended” and “Since the bedsore risk is high, posture change and introduction of a bedsore prevention mattress are recommended”. Specifically, the care plan AI determination unit of the present system inputs multidimensional time-series data extracted from the nursing care record management unit to a preprocessing module. The preprocessing module normalizes continuous values such as vital data and ADL scores, and converts categorical variables such as care content and accident history into multidimensional embedding vectors. The present system combines these vectors and shapes them into a tensor format that can be input to a large language model. The input data to the model includes, for example, a numerical array of vital transitions over the past three months, timestamps of fall histories, and structured texts such as “ADL score 20 points” and “cognitive function score 10 points”. The number of dimensions of the input tensor of the model is, for example, a third-order tensor consisting of a batch size, a sequence length, and a feature dimension. The model applies a self-attention mechanism to the input tensor to extract a complex causal relationship between a past event and a current state. The output data from the model is a probability distribution vector indicating a category of a recommended care plan, and a natural language text explaining the basis thereof. Specific examples of the output of the model include score data such as “probability of physical assistance enhancement 0.95, probability of dementia care enhancement 0.88”, and a basis text such as “Since two falls have occurred in the past three months and the risk when moving to a toilet at night is high, enhancement of a night watching over system is recommended”. The present system selects a care plan having the maximum output probability score as an optimal solution. The present system transfers the selected care plan and basis text to a subsequent explanation generation unit. The present system models tacit knowledge of a skilled care manager as a weight parameter of a neural network to realize objective and data-driven care plan creation. The present system brings about a technical effect of significantly shortening the time required for care plan creation and reducing the work load of staff. The present technology is applicable to use cases such as assessment work for a new user and regular review work of a care plan accompanying a state change. In learning of the model, the present system uses a pair of an excellent past care plan and user state data as teacher data, and updates weights so as to minimize a cross-entropy error.

The explanation generation unit automatically generates an explanatory text in a natural language easy for a user or a family member to understand using generative AI based on the care plan and the basis explanation generated by the care plan AI determination unit. For example, a prompt text such as “Based on this care plan and basis, please create an explanatory text that is easy for the user himself/herself to understand and accept”, “Please explain including a care policy and lifestyle guidance in expressions easy for an elderly family member to understand”, or “Please explain including future points to note and points of watching over for a family member of a user with dementia” is given to the generative AI, and an explanatory text considering the age, cognitive function, family structure, understanding level, language setting, and the like of the user is generated. For example, in the case of physical assistance enhancement, an explanatory text such as “Since it is difficult for the user to perform activities of daily living, a caregiver will be attached to all of meals, bathing, excretion, changing clothes, and the like. While considering safety, we will also provide support to encourage independence as much as possible” is generated. In the case of dementia care enhancement, an explanatory text such as “Since a decline in cognitive function is observed, we will perform watching over, calling out, and environmental adjustment so that the user can spend time with peace of mind. If wandering or confusion is observed, we will also thoroughly implement measures to prevent falls and accidents” is generated. In the case of fall prevention enhancement, an explanatory text such as “Since there is a fall history, we will thoroughly implement watching over and environmental improvement for fall prevention. We will also consider eliminating steps on the floor, installing handrails, and utilizing walking aids” is generated. In the case of medication management support, an explanatory text such as “Since medication errors continue, we will utilize a medication support device and a checklist, and staff will always check at the time of medication” is generated. The explanation generation unit can provide the generated explanatory text by various methods such as a nursing care staff terminal, a portal site for family members, printed matter, and e-mail. Specifically, the explanation generation unit of the present system receives specialized care plan data output from the care plan AI determination unit. The generation unit combines the received data and a context vector including attribute information of the user and the family member. The generation unit inputs the combined data to a large language model specialized for natural language generation. The input data to the model includes, for example, structured data such as “Recommended plan: Fall prevention enhancement, Basis: Two falls in the past three months”, and control parameters such as “Target person: 80-year-old family member, Understanding level: Plain, Tone: Giving a sense of security”. The model converts specialized nursing care terms into general vocabulary according to the input control parameters. The model executes an autoregressive decoding process to generate a natural explanatory text along the context. The output data from the model is character string data optimized for a specified persona. Specific examples of the output of the model include specific and considerate texts such as “Recently, there was a case where the user lost balance when going to the restroom at night” and “In order for the user to spend time safely, we will install a handrail next to the bed, and staff will regularly call out at night”. The present system outputs the generated text data to a subsequent distribution module. The distribution module writes the text to a database of the portal site for family members, and simultaneously transmits a push notification to a smartphone of the family member. The present system reduces the time for staff to individually devise an explanatory text, and standardizes the quality of communication. The present system exhibits a technical effect of reducing anxiety of family members and improving reliability of nursing care services through automatic generation by a computer. The present technology is extremely effective in use cases such as reporting to family members at the time of monthly care plan update and rapid situation explanation when an accident occurs.

The data linkage unit has a function of encrypting the nursing care record data recorded by the nursing care record management unit and enabling secure sharing with another nursing care facility or medical institution. For example, when a user transfers to another facility or hospital, a server of a transfer source facility encrypts a nursing care record history of the user and transmits it to a server of a transfer destination facility or medical institution via a secure communication path. At the transfer destination, the server side can decrypt the nursing care history of the user and integrate it into its own system. Furthermore, even when sharing information with an external medical institution or administrative agency, only necessary information can be summarized and anonymized for sharing. For example, a prompt text such as “In order to share the nursing care history of this user with another facility, please summarize necessary information and output it in a form that considers personal information protection” is given to the generative AI, and the summary and anonymization of the shared data are also automatically performed. For example, at the time of a nursing care insurance certification survey, an administrative report, or linkage with a medical institution, main care content, accident history, rehabilitation progress, medication status, request history from family members, and the like for the past one year are summarized, and only a necessary range can be securely shared. Specifically, the data linkage unit of the present system receives a data sharing request from an external system. The linkage unit verifies an authentication token included in the request to confirm an access authority. When the authority is confirmed, the linkage unit extracts the nursing care record data of the target user from the database. The linkage unit executes a summary and anonymization process using the generative AI on the extracted data. The input data to the AI is, for example, all nursing care record texts for the past one year, an institution type of a sharing destination (medical institution, administrative agency, etc.), and a prompt character string specifying an extraction requirement. The AI extracts information matching the needs of the sharing destination from the input data, and masks unnecessary personal information. The output data from the AI is structured text data for which anonymization and summary have been completed. Specific examples of the output of the AI include summary data such as “Number of falls in the past one year: 3 times”, “Most recent ADL score: 45 points”, and “Currently implemented rehabilitation: Walking training twice a week”. The present system inputs the output summary data to a subsequent encryption module. The encryption module encrypts the data in block units using an advanced encryption standard algorithm. The present system transmits the encrypted data to a server of the external system via a secure communication protocol. The present system completely erases a temporary encryption key from a memory after the transmission is completed. The present system mathematically eliminates a data leakage risk on a communication path while ensuring data interoperability between different systems. The present system brings about a technical effect of achieving both safety and efficiency of data transfer. The present technology becomes an indispensable basic technology in use cases such as inter-facility information sharing when a user uses a day service and a short stay together, and rapid information provision to a medical institution at the time of emergency transport.

The falsification detection unit has a function of verifying the hash chain of the nursing care record data and automatically detecting the presence or absence of falsification. For example, when a past nursing care record is illegally rewritten, a falsified part is specified by verifying the hash chain, and a warning is notified to an administrator or a nursing care worker. Furthermore, the generative AI is used to analyze whether an unnatural change or an abnormal pattern is included in the content of the nursing care record data, and a pattern with a high possibility of falsification or fraud, such as a case where duplicate records by a plurality of different staff exist on the same day, a case where a plurality of records are changed at once in a short period of time, or a case where there is a contradiction between care content and a user state, is automatically detected, and warning information is generated and notified. For example, a prompt text such as “Please analyze in detail from a nursing care viewpoint whether there is an unnatural change or an abnormal pattern in this nursing care history, and point out any suspicious part” is given to the generative AI to perform the analysis. Furthermore, at the time of audit response or administrative investigation, the presence or absence of falsification of all past records can be collectively verified and output as an audit trail. Specifically, the falsification detection unit of the present system operates as a regular batch process or as a real-time process at the time of data access. The detection unit sequentially reads the hash chain of the nursing care records stored in the database from the first block. The detection unit combines the data content of each record and the hash value of the immediately preceding record, and calculates a new hash value using a secure hash algorithm. The detection unit compares the calculated hash value with the recorded hash value, and immediately transmits an alert to the system administrator when a mismatch is detected. In addition to this cryptographic verification, the present system executes semantic anomaly detection using the generative AI. The present system inputs time-series nursing care record texts and metadata to a large language model. The input data to the model is, for example, shift information of staff for the past one week, timestamps of record input by each staff, and text vectors of record content. The model maps the data to a latent space in which a standard pattern of nursing care operations is learned from the input data, and calculates an anomaly score. The output data from the model is an anomaly probability score for each record, and text data indicating a reason determined to be abnormal. Specific examples of the output of the model include analysis results such as “Score 0.98 because assistance records by the same staff exist at two physically separated places in the same time zone” and “Score 0.95 because the ADL score of the user has rapidly improved from the previous day and is medically unnatural”. When the output anomaly score exceeds a threshold value, the present system isolates the corresponding record and generates a detailed audit log. The present system contextually discovers a false record deviating from actual business conditions, which is difficult to detect by a simple rule base. The present system exhibits a technical effect of dramatically improving the reliability of nursing care records. The present technology is applicable to use cases such as voluntary inspection before on-site guidance by an administration and prevention of fraudulent billing of nursing care rewards.

In addition, the care plan AI determination unit and the nursing care record management unit also have a function of automatically generating a summary text of the nursing care record data using generative AI. For example, a prompt text such as “Please summarize the nursing care history of this user over the past year, and briefly summarize main care contents, change points, and risk factors” is given to the generative AI to enable a nursing care worker to grasp the entire picture of the user in a short time. The summary text is recorded as a part of the nursing care history, and can be viewed on a staff terminal or a family terminal. For example, a summary briefly summarizing main points, such as “three falls in the past year, rehabilitation progress shows an increase in walking distance from 50 m to 100 m, medication errors occur once a month, and cognitive function tends to decline”, is automatically generated. Specifically, the nursing care record management unit of the present system extracts all nursing care record data of a target user for a specified period (for example, the past year) from a database. The management unit combines the extracted huge amount of text data and numerical data in chronological order, and preprocesses them into a format that can be input to a large language model. The present system divides the preprocessed data into chunk units and inputs them to the large language model. Input data to the model is, for example, an array of daily vital sign transition data, texts of accident reports, and rehabilitation implementation records. The model applies a self-attention mechanism to the input data to extract trends of state changes and important events over a long period of time. Output data from the model is concise summary text data integrating the extracted important information. Specific examples of the output of the model include structured summary texts such as “three falls occurred in the past year, but there were no serious injuries such as fractures”, “walking distance increased from 50 m to 100 m due to continuation of rehabilitation, and improvement in lower limb muscle strength is seen”, and “cognitive function tends to decline gently, and medication errors occur about once a month”. The present system transfers the generated summary text to a subsequent display processing module. The display processing module highlights and displays the summary text at the top of a dashboard screen of the staff terminal. The present system dramatically reduces the time for staff to go back and view a huge amount of past records. The present system brings about a technical effect of reducing consumption of computer resources required for information retrieval and improving responsiveness of the entire system. The present technology is extremely useful in use cases such as streamlining handover work to night shift staff and automatically creating user state report materials in person-in-charge meetings. In learning of the model, the present system uses summary reports created by skilled staff in the past as training data to fine-tune the summary generation capability of the model.

As described above, the embodiment of the present invention integrally and specifically realizes various functions required in a nursing care site, such as recording, management, and falsification prevention of nursing care records, care plan determination and basis explanation by AI, explanation text generation for users and families, secure data linkage between nursing care facilities, falsification detection, and nursing care record summarization, with generative AI as an essential element, and significantly improves transparency, reliability, explainability, efficiency, and safety of the nursing care site, and is an extremely practical and highly scalable system that can be widely utilized in all nursing care scenes such as daily care, rehabilitation, accident response, family explanation, inter-facility linkage, audit response, and administrative reporting. Specifically, the present system functions as a comprehensive information processing platform integrating a distributed database, an advanced encryption module, and a large language model. The present system implements each functional module as a microservice architecture to ensure availability and scalability of the entire system. The present system seamlessly links construction of an immutable data store using a hash chain by the nursing care record management unit, integration of inference in a high-dimensional vector space and natural language generation by the care plan AI determination unit, dynamic text rendering according to a user context by the explanation generation unit, secure data routing using a public key infrastructure by the data linkage unit, and hybrid processing of cryptographic verification and semantic anomaly detection by the falsification detection unit. In these processes, the present system ensures real-time response performance by offloading an inference pipeline of the large language model to parallel computing cores of a graphics processing unit. The present system dramatically reduces the administrative workload of nursing care staff by entrusting a computer with processing of a huge amount of nursing care data exceeding the limits of human cognitive ability. The present system brings about an improvement in computer technology itself, which enables provision of data-driven high-quality nursing care services, not limited to mere digitization of business. The present system exerts complex technical effects of preventing nursing care accidents, facilitating communication with families, and optimally allocating nursing care resources. The present technology is an extremely innovative solution that demonstrates its true value in large-scale use cases such as construction of a nationwide nursing care provider network infrastructure system and a municipality-level nursing care information linkage platform.

### System Configuration

The system according to the present embodiment comprises a nursing care record management unit, a care plan AI determination unit, an explanation generation unit, a data linkage unit, and a falsification detection unit. The nursing care record management unit has a function of recording and managing nursing care record data for each user in chronological order, receives all nursing care-related data such as meal assistance, excretion assistance, bathing assistance, transfer assistance, medication management, rehabilitation implementation, recreation participation status, vital measurement, cognitive function evaluation, ADL evaluation, fall history, accident/near-miss report, user request/complaint, communication matter from family, medical treatment, outing/overnight stay record, and emergency transport record input by staff from a tablet terminal or a personal computer terminal, for example, assigns user identification information, care content, vital data, a cognitive function score, an ADL score, a fall history, a recording time, and a hash value of an immediately preceding record, and stores them in a database on a server. For example, in meal assistance at breakfast, “meal assistance implemented”, “intake 80%”, “no choking”, “post-meal medication support”, etc. are recorded, in rehabilitation in the morning, “walking training 20 minutes”, “walking distance 50 m”, “no fall”, “mild fatigue”, etc. are recorded, in bathing assistance in the afternoon, “bathing assistance implemented”, “no skin condition abnormality”, “watching when entering and exiting bathtub”, “low fall risk”, etc. are recorded, and in excretion assistance and night watching at night, “excretion assistance twice”, “night awakening once”, “good sleep”, etc. are recorded. All of these records are managed in chronological order, and each record is assigned a hash value of an immediately preceding record, whereby the records are stored as a hash chain, and data recorded at a plurality of facilities are also integrally managed. Specifically, the nursing care record management unit of the present system receives a wide variety of nursing care data asynchronously transmitted from a plurality of input interfaces in real time. The management unit classifies the received data into structured data and unstructured data, and normalizes them according to a unified data schema. The management unit inserts event data such as meal assistance and excretion assistance into the database together with a time stamp. The management unit expands numerical data such as vital measurement values and cognitive function scores on a memory as an array of floating-point numbers. When generating a data record, the present system calculates a cryptographic hash value of an immediately preceding recorded record and assigns it to a header of a current record. By this processing, the present system connects all nursing care records as a hash chain to ensure sequence and integrity of the data. The present system also merges data transmitted from a plurality of facilities onto a single logical timeline using a user identifier as a key. The present system adds, for example, a meal assistance record at breakfast, a rehabilitation record in the morning, a bathing assistance record in the afternoon, and an excretion assistance record at night to the hash chain as independent blocks, respectively. With this data structure, the present system mathematically guarantees data integrity and traceability even in an environment where writing occurs from a plurality of nodes. The present system brings about a technical effect of realizing high-throughput data recording while avoiding lock contention of the database. The present system dramatically increases reliability as an audit trail by making it physically difficult to rewrite or delete data. The present technology plays an extremely important role in use cases such as comprehensive state grasping of a user who uses a plurality of nursing care services in combination and rapid cause investigation when a nursing care accident occurs. The present system constructs a data pipeline that seamlessly provides the accumulated chronological data as input data for the subsequent care plan AI determination unit and falsification detection unit.

The care plan AI determination unit has a function of automatically determining an optimal care plan by analyzing the latest data and past history data of the user acquired from the nursing care record management unit using generative AI. For example, when the ADL score is low and the cognitive function score is also low, a complex care policy of “physical assistance enhancement+dementia care enhancement” is presented, and a basis explanation such as “since the ADL score is 20 points or less and almost full assistance is required, and the cognitive function score is 10 points or less and severe dementia is suspected, a two-caregiver system is recommended for all of meals, excretion, bathing, changing clothes, and transfer, and orientation training, environmental adjustment, and wandering countermeasures are enhanced as dementia care” is generated. When there are a plurality of fall histories, “fall prevention enhancement+environmental improvement+walking training enhancement” is set, and an explanation such as “since there were two falls in the past three months and many falls during night toilet movement, installation of night lighting, securing a flow line to the toilet, introduction of a walking aid, and enhancement of a night watching system are recommended” is output. When medication errors occur frequently, “medication management support enhancement” is set, and a basis such as “since medication errors occur twice or more a week, introduction of a medication support device, calling out during medication, and utilization of a medication checklist are recommended” is shown. When rehabilitation progress is stagnant, “rehabilitation program review” is set, and an explanation such as “since the walking distance has not increased in the past month, reevaluation of rehabilitation contents and new goal setting are recommended” is generated. Furthermore, referring to the living environment, family structure, medical history, presence or absence of medical treatment, nursing care insurance service utilization status, etc. of the user, highly individualized care plans and bases are output from multiple angles, such as “since the user is an elderly person living alone and watching is insufficient, introduction of an emergency call device is recommended” and “since the bedsore risk is high, posture change and introduction of a bedsore prevention mattress are recommended”. Specific examples of the prompt text given to the generative AI include “Based on information such as ADL score, cognitive function score, fall history, vital data, medical history, medication status, rehabilitation progress, family structure, and living environment, please explain in detail an optimal care plan for this user and its basis from medical and nursing care perspectives”. Specifically, the care plan AI determination unit adopts a hybrid architecture combining a large language model having billions of parameters and a graph neural network. The care plan AI determination unit receives a multidimensional tensor indicating the state of the user as input data. The multidimensional tensor includes, for example, a numerical vector obtained by normalizing the ADL score and the cognitive function score, a chronological array of past fall histories and vital signs, and a feature amount obtained by embedding and vectorizing a nursing care record in a text format by a natural language processing model. The care plan AI determination unit executes interpolation processing of missing values and smoothing processing of outliers as preprocessing. Specific examples of the input data to the care plan AI determination unit include, first, a numerical vector in which the ADL score is 15 points and the cognitive function score is 8 points. Second, a chronological array in which the number of falls in the past three months is two and the number of awakenings at night is three on average is given. Third, a text embedding vector indicating that the meal intake tends to decrease is given. The care plan AI determination unit inputs these input tensors to a transformer layer equipped with a self-attention mechanism. The transformer layer calculates a hidden correlation between the respective feature amounts in a high-dimensional space. The care plan AI determination unit generates a probability distribution vector indicating a category of a recommended care plan and a token sequence of a natural language constituting the basis thereof as output data. Specific examples of the output data include, first, a score value indicating that the probability of the physical assistance enhancement category is 95 percent in percentage. Second, a score value indicating that the probability of the dementia care enhancement category is 88 percent in percentage is given. Third, structured text data recommending enhancement of the night watching system is given. The care plan AI determination unit determines whether the generated probability distribution vector exceeds a preset threshold value as subsequent processing. The care plan AI determination unit determines a care plan category exceeding the threshold value, and transmits it to the subsequent explanation generation unit together with a corresponding basis text. In a learning phase, the care plan AI determination unit uses millions of care plan data created by excellent care managers in the past as training data. The care plan AI determination unit optimizes weights of the model using a cross-entropy error function. With this configuration, the present system replaces care plan creation work, which has relied on human rules of thumb, with arithmetic processing in a high-dimensional space based on objective data. As a result, the present system exerts a technical effect of significantly shortening the care plan creation time and comprehensively extracting risk factors that tend to be overlooked. The present technology is widely applicable to care management work in various nursing care facilities such as special nursing homes for the elderly and day service centers.

The explanation generation unit has a function of automatically generating an explanatory text in a natural language easy for a user or a family member to understand using generative AI based on the care plan and the basis explanation generated by the care plan AI determination unit. For example, prompt texts such as “Based on this care plan and basis, please create an explanatory text that is easy for the user himself/herself to understand and accept”, “Please explain including a care policy and lifestyle guidance in expressions easy for elderly family members to understand”, and “Please explain for family members of a user with dementia, including future points to note and points for watching” are given to the generative AI to generate an explanatory text considering the user's age, cognitive function, family structure, level of understanding, language setting, and the like. For example, in the case of physical assistance enhancement, an explanatory text such as “Since it is difficult for the user to perform activities of daily living, a caregiver will be attached to all of meals, bathing, excretion, changing clothes, etc. While considering safety, we will also provide support to encourage independence as much as possible” is generated. In the case of dementia care enhancement, an explanatory text such as “Since a decline in cognitive function is seen, we will perform watching, calling out, and environmental adjustment so that the user can spend time with peace of mind. If wandering or confusion is seen, we will also thoroughly implement countermeasures to prevent falls and accidents” is generated. In the case of fall prevention enhancement, an explanatory text such as “Since there is a fall history, we will thoroughly implement watching and environmental improvement for fall prevention. We will also consider eliminating steps on the floor, installing handrails, and utilizing walking aids” is generated. In the case of medication management support, an explanatory text such as “Since medication errors continue, we will utilize a medication support device and a checklist, and staff will always check at the time of medication” is generated. Specifically, the explanation generation unit includes a large language model specialized for conditional text generation. The explanation generation unit receives structured data of the care plan output from the preceding care plan AI determination unit and a context vector indicating attribute information of the user as input data. The context vector is a multidimensional array obtained by digitizing or one-hot encoding the user's age, cognitive function level, family's nursing care capability, and desired language setting. Specific examples of the input data to the explanation generation unit include, first, a care plan code instructing physical assistance enhancement and a numerical vector indicating that the user's age is 85 years old. Second, an attribute matrix indicating that the cognitive function score is low and the family lives far away is given. Third, a control flag array requesting plain expressions avoiding technical terms is given. The explanation generation unit dynamically embeds these input data into a prompt template and inputs them to a transformer-based decoder network. The decoder network executes autoregressive inference processing and sequentially calculates appearance probabilities of words. The explanation generation unit outputs a token sequence of an explanatory text composed of a natural language and an attention score indicating the importance of each sentence as output data. Specific examples of the output data include, first, plain text data explaining that a caregiver will be attached to meals and bathing. Second, a structured text in which specific environmental improvement procedures for fall prevention are itemized is given. Third, text data explaining night watching points for families is given. The explanation generation unit executes filtering processing of inappropriate terms on the generated text data as subsequent processing. The explanation generation unit transfers the text data that has passed the filtering processing to a display module of a staff terminal or a distribution server of a portal site for families. The explanation generation unit improves naturalness and accuracy of the generated text by reflecting human feedback through fine-tuning using reinforcement learning. With this configuration, the present system realizes unconventional data conversion processing of automatically converting specialized medical and nursing care terms into plain expressions. As a result, the present system exerts a technical effect of dramatically improving accuracy of information transmission while optimizing consumption of computer resources required for creating explanation materials. The present technology is applicable to an informed consent support system for users and families in visiting nursing care stations and comprehensive community support centers.

The data linkage unit has a function of encrypting the nursing care record data recorded by the nursing care record management unit and enabling secure sharing with another nursing care facility or medical institution. For example, when a user transfers to another facility or hospital, a server of a transfer source facility encrypts a nursing care record history of the user and transmits it to a server of a transfer destination facility or medical institution via a secure communication path. In the transfer destination, the server side can decrypt the nursing care history of the user and integrate it into its own system. Furthermore, even when sharing information with an external medical institution or administrative agency, only necessary information can be summarized and anonymized for sharing. For example, a prompt text such as “In order to share the nursing care history of this user with another facility, please summarize necessary information and output it in a form that considers personal information protection” is given to the generative AI to automatically perform summarization and anonymization of shared data. For example, at the time of nursing care insurance certification investigation, administrative reporting, or linkage with a medical institution, main care contents, accident history, rehabilitation progress, medication status, request history from family, etc. for the past year are summarized, and only a necessary range can be securely shared. Specifically, the data linkage unit functions as a secure data processing module integrating an advanced encryption algorithm and a natural language processing model. The data linkage unit receives raw text data accumulated in the nursing care record management unit and an access control matrix indicating an authority level of a sharing destination as input data. Specific examples of the input data to the data linkage unit include, first, unstructured text data of tens of thousands of characters including all nursing care records for the past year. Second, an authority flag vector indicating that the sharing destination is an external medical institution is given. Third, an array of an extraction item list designated for reporting to an administrative agency is given. The data linkage unit extracts personally identifiable information from the input text data as preprocessing. The data linkage unit automatically masks confidential information such as a name and an address using a bidirectional recurrent neural network specialized for named entity extraction. The data linkage unit inputs the masked text data to extraction-type and generation-type summarization models. The summarization models compress a data size while retaining an important context. The data linkage unit outputs text data subjected to anonymization and summarization processing and a digital signature proving integrity of the data as output data. Specific examples of the output data include, first, a summary text of several hundred characters extracting only the fall history and rehabilitation progress for the past year. Second, an anonymized data set in which personal information is replaced with asterisk symbols is given. Third, an encrypted token of a temporary access key necessary for decryption in a sharing destination system is given. The data linkage unit encrypts the output summary data using a common key cryptosystem as subsequent processing. The data linkage unit transmits the encrypted payload to an external system via a secure communication protocol. With this configuration, the present system executes computer-specific processing of dynamic data conversion and encryption according to the authority of the sharing destination, rather than mere data transmission and reception. As a result, the present system exerts a technical effect of mathematically eliminating an information leakage risk while minimizing consumption of a network bandwidth. The present technology is applicable to a wide-area medical linkage network that securely exchanges highly confidential personal health records among medical institutions, nursing care facilities, and administrative agencies.

The falsification detection unit has a function of automatically detecting presence or absence of falsification by verifying the hash chain of the nursing care record data. For example, when a past nursing care record is illegally rewritten, a falsified part is specified by verifying the hash chain, and a warning is notified to an administrator or a nursing care worker. Furthermore, the generative AI is used to analyze whether an unnatural change or an abnormal pattern is included in the content of the nursing care record data, and automatically detects a pattern with a high possibility of falsification or fraud, such as when duplicate records by a plurality of different staff exist on the same day, when a plurality of records are changed at once in a short period of time, or when there is a contradiction between care content and a user state, and generates and notifies warning information. For example, a prompt text such as “Please analyze in detail from a nursing care perspective whether there is an unnatural change or an abnormal pattern in this nursing care history, and point out any suspicious parts” is given to the generative AI to perform analysis. Furthermore, at the time of audit response or administrative investigation, presence or absence of falsification of all past records can be collectively verified and output as an audit trail. Specifically, the falsification detection unit comprises a hybrid verification engine combining a cryptographic hash function and a machine learning model specialized for anomaly detection. The falsification detection unit receives chronological log data of nursing care records extracted from the database and an array of hash values accompanying each log as input data. Specific examples of the input data to the falsification detection unit include, first, a multidimensional vector of metadata including a creation time, an update time, and a creator ID of a record. Second, a 256-bit hash value array by a SHA256 algorithm calculated from an immediately preceding record is given. Third, a composite tensor including a text embedding vector of record content and numerical data of vital signs is given. As first processing, the falsification detection unit recalculates a hash value from the input log data, and executes matching processing with the stored hash chain at high speed. As second processing, the falsification detection unit inputs the chronological log data to an autoencoder-based anomaly detection model. The anomaly detection model has learned a latent space representation of a normal recording pattern, and calculates a reconstruction error between the input data and reconstructed data. The falsification detection unit outputs a boolean flag indicating a verification result of the hash chain and a continuous anomaly score indicating a degree of anomaly of each log data as output data. Specific examples of the output data include, first, an error code indicating a mismatch of hash values in a specific block. Second, an anomaly score value in a range of 0 to 1 indicating that records were created at physically distant locations at the same time is given. Third, a warning label indicating that a logical contradiction exists between the care content and the ADL score of the user is given. As subsequent processing, when the anomaly score exceeds a preset threshold value, the falsification detection unit generates an alert packet including an identifier of the corresponding log and a reason for the anomaly. The falsification detection unit immediately transmits the alert packet to a monitoring terminal of a system administrator. With this configuration, the present system automatically detects minute data contradictions and highly disguised falsifications, which are difficult to find by human visual inspection, by feature amount analysis in a high-dimensional space. As a result, the present system exerts a technical effect of mathematically and objectively ensuring evidence capability and authenticity of electronic records while reducing human costs required for audit work. The present technology is widely applicable to electronic medical record systems in the medical and nursing care fields where strict compliance is required, and audit log monitoring systems of financial institutions.

As described above, the system according to the present embodiment comprises the nursing care record management unit, the care plan AI determination unit, the explanation generation unit, the data linkage unit, and the falsification detection unit, and each unit utilizes generative AI to integrally and specifically realize various functions such as recording, management, diagnosis, explanation, sharing, falsification detection, and summarization of nursing care records, thereby dramatically improving transparency, reliability, explainability, safety, and efficiency of the nursing care site. Specifically, the present system adopts a distributed computing architecture in which a plurality of independent microservices cooperate via asynchronous communication. The present system containerizes each processing unit and dynamically allocates computing resources using an orchestration tool. The present system receives metrics data indicating an operating status of the entire system and a chronological tensor indicating a communication traffic volume between the respective processing units as input data. Specific examples of the input data to the present system include, first, a numerical vector indicating a CPU usage rate and memory consumption of the care plan AI determination unit. Second, a chronological array in milliseconds indicating latency of encryption processing in the data linkage unit is given. Third, integer value data indicating a length of a queue waiting for processing by the falsification detection unit is given. The present system inputs these input data to a load balancing module. The load balancing module calculates optimal resource allocation using a reinforcement learning-based routing algorithm. The present system outputs a control command instructing scale-out or scale-in of each container and update information of a traffic routing table as output data. Specific examples of the output data include, first, a scaling command to double the number of instances of the explanation generation unit. Second, update data of a network policy restricting communication to a specific node is given. Third, a health check score indicating soundness of the entire system is given. As subsequent processing, the present system transmits the output control command to an infrastructure management API to change settings of a physical server group in real time. With this configuration, the present system realizes an unconventional processing method of autonomously optimizing performance of the entire system even for sudden access concentration or large-scale data processing requests. As a result, the present system exerts a technical effect of eliminating a single point of failure, enabling 24/7 non-stop operation, and maximizing processing throughput of the entire system. The present technology is applicable to a large-scale cloud-type nursing care business support platform deployed on a nationwide scale and a medical information network infrastructure requiring real-time performance.

### Implementation Steps

Step 1: Collection and chronological management of nursing care record data. A nursing care staff inputs nursing care-related data such as meal assistance, excretion assistance, bathing assistance, transfer assistance, medication management, rehabilitation implementation, recreation participation status, vital measurement, cognitive function evaluation, ADL evaluation, fall history, accident/near-miss report, user request/complaint, contact matter from a family member, medical treatment, outing/overnight stay record, and emergency transportation record for each user using a tablet terminal or a personal computer terminal, and the nursing care record management unit stores these data in chronological order in a database on a server together with user identification information, care content, vital data, a cognitive function score, an ADL score, a fall history, a recording time, and a hash value of an immediately preceding record, and connects each record as a hash chain, thereby ensuring authenticity and falsification prevention of the record. Specifically, in this step, the system executes an asynchronous data synchronization mechanism between an edge device and a cloud server. The system receives raw multimodal data acquired from various sensors and an input interface of the edge device as input data. As a specific example of the input data to the system, first, structured JSON data indicating a meal intake amount and an excretion frequency input from a touch panel of the tablet terminal can be cited. Second, a chronological numerical array of a heart rate and a blood pressure continuously transmitted from a wearable device via Bluetooth communication can be cited. Third, voice waveform data of the nursing care staff acquired from a voice input microphone can be cited. As preprocessing, the system inputs the voice waveform data to a voice recognition model and converts it into text data. The system further assigns a time stamp to all the input data and executes a data cleansing process. As output data, the system outputs normalized record data for database storage and a new cryptographic hash value calculated including the hash value of the immediately preceding record. As a specific example of the output data, first, row data of a relational database in which a user ID, a recording time, and care content are mapped according to a predetermined schema can be cited. Second, a hash value consisting of a 64-character hexadecimal character string generated by a SHA256 algorithm can be cited. Third, a status code indicating a synchronization state of data can be cited. As subsequent processing, the system writes the generated record data and hash value to a storage node of a distributed database, and completes a transaction commit process. With this configuration, the system realizes a unique data management method of temporarily storing data on the edge side even in an environment where a network connection is unstable, and performing differential synchronization when communication is restored. As a result, the system exhibits a technical effect of imparting mathematical falsification resistance by a hash chain structure to the entire system while completely preventing data loss. This technology is widely applicable to a mobile healthcare area where work in an offline environment frequently occurs, such as home-visit nursing care and home-visit nursing.

Step 2: Optimal care plan determination and basis explanation generation by care plan AI. The care plan AI determination unit inputs the latest data and past history data of the user acquired from the nursing care record management unit to the generative AI, gives a prompt sentence “Please explain in detail an optimal care plan for this user and a basis thereof from medical and nursing care viewpoints based on information such as an ADL score, a cognitive function score, a fall history, vital data, a medical history, a medication status, a rehabilitation progress, a family structure, and a living environment” to the generative AI to explain in detail the optimal care plan for this user and the basis thereof from the medical and nursing care viewpoints based on the information such as the ADL score, the cognitive function score, the fall history, the vital data, the medical history, the medication status, the rehabilitation progress, the family structure, and the living environment, and automatically generates a complex care policy and a basis explanation such as physical assistance enhancement, dementia care enhancement, fall prevention enhancement, medication management support, and rehabilitation program review. Specifically, in this step, the system executes an advanced inference process using a retrieval-augmented generation architecture. The system receives, as input data, a feature vector indicating the latest state of the user extracted from the database, and a retrieval result from a vector database storing past similar cases. As a specific example of the input data to the system, first, a chronological gradient vector indicating a downward trend of the ADL score can be cited. Second, an embedding vector of a past care plan successfully applied to a user in a similar state, extracted from the vector database by cosine similarity calculation, can be cited. Third, a token sequence of a system prompt for an inference instruction automatically generated by the system can be cited. As preprocessing, the system combines these input data into one huge context window and converts it into a tensor format suitable for an input layer of a transformer model. As output data, the system outputs a certainty score for each element of the recommended care plan, and a natural language text stream explaining the medical basis thereof. As a specific example of the output data, first, a certainty score value of 0.95 indicating the necessity of physical assistance enhancement can be cited. Second, a structured JSON object describing a specific procedure for dementia care enhancement can be cited. Third, a flag variable warning of an increase in fall risk can be cited. As subsequent processing, the system verifies whether the output certainty score satisfies a predetermined threshold, and if it satisfies the threshold, stores the generated care plan in the database as confirmed data. In the inference process, the system dynamically weights an important feature amount in the input data by a self-attention mechanism. With this configuration, the system executes a computer-specific process of probabilistic inference in a high-dimensional space based on millions of parameters, rather than a simple rule-based conditional branch. As a result, the system exhibits a technical effect of simultaneously considering an enormous number of variables exceeding human cognitive limits and instantaneously generating an individually optimized and highly accurate care plan. This technology is applicable to care management for elderly people with complex diseases and a treatment plan formulation support system in personalized medicine.

Step 3: Automatic generation of explanatory text for user and family member. The explanation generation unit gives prompt sentences such as “Please create an explanatory text that is easy for the user to understand based on this care plan and basis”, “Please explain including a care policy and life guidance in an expression easy for an elderly family member to understand”, and “Please explain including future precautions and monitoring points for a family member of a user with dementia” to the generative AI based on the care plan and the basis explanation generated by the care plan AI determination unit, automatically generates an explanatory text considering the user's age, cognitive function, family structure, understanding level, language setting, and the like, and provides it by various methods such as a staff terminal, a family portal site, printed matter, and an e-mail. Specifically, in this step, the system executes a dynamic natural language conversion process according to an attribute of a target audience. The system receives, as input data, structured data of the confirmed care plan and an attribute vector defining a persona of the user or the family member who is an explanation target person. As a specific example of the input data to the system, first, original text data of the care plan including technical medical terms can be cited. Second, an array of categorical variables indicating an age group, IT literacy, and a desired language of the explanation target person can be cited. Third, an emotion parameter vector for controlling a tone and manner of the output text can be cited. As preprocessing, the system selects an optimal prompt template based on the attribute vector, and inserts the care plan data into a placeholder in the template. As output data, the system outputs text data of the explanatory text optimized for the target person, and reading metadata for voice synthesis. As a specific example of the output data, first, a token sequence of a plain sentence in which technical terms are replaced with general vocabulary can be cited. Second, a markup text to which an HTML tag for emphasizing an important point is added can be cited. Third, tag data in an SSML format for controlling an utterance speed and intonation in a voice synthesis engine can be cited. As subsequent processing, the system routes the generated text data to a PDF generation module, an e-mail transmission module, or a voice synthesis module. In a decoding process of a language model, the system controls a balance between diversity and accuracy of the generated sentence by adjusting a temperature parameter and top-P sampling. With this configuration, the system realizes a non-conventional data conversion method of dynamically generating various output formats from the same original data. As a result, the system exhibits a technical effect of completely automating personalization of information transmission, dramatically reducing a document creation burden on the nursing care staff, and improving the understanding level of the user and the family member. This technology is applicable to an automatic business manual generation system for foreign workers requiring multilingual support, and a user interface of a medication guidance system for patients.

Step 4: Summary generation of nursing care record data. The care plan AI determination unit and the nursing care record management unit give a prompt sentence such as “Please summarize a nursing care history of this user for the past one year, and briefly summarize main care content, change points, and risk factors” to the generative AI, extract the number of falls, rehabilitation progress, medication error occurrence frequency, change in cognitive function, transition of ADL score, accident history, request history from a family member, and the like as main points, and automatically generate a summary text enabling a nursing care worker or a family member to grasp the entire picture of the user in a short time, so that it can be stored and viewed as a part of the record. Specifically, in this step, the system executes advanced information compression and feature extraction processing for long chronological text data. The system receives, as input data, a text data group of all nursing care records accumulated within a specified period, and a query vector specifying a viewpoint of the summary. As a specific example of the input data to the system, first, huge character string data obtained by combining thousands of daily business report texts created in the past one year in chronological order can be cited. Second, an embedding vector indicating a specific extraction theme such as a fall risk or a change in cognitive function can be cited. Third, an integer value parameter limiting the maximum number of characters of the output summary text can be cited. As preprocessing, the system divides the input long text data into semantic paragraphs, and calculates an importance score of each paragraph using a TF-IDF algorithm or a sentence embedding model. As output data, the system outputs summary text data obtained by extracting and reconstructing highly important information, and metadata including link information to the original record that is the basis of the summary. As a specific example of the output data, first, structured text data in which main change points in the past one year are itemized in chronological order can be cited. Second, a list array in which extracted risk factors are sorted in order of importance can be cited. Third, index mapping data indicating which original record each sentence of the summary text is derived from can be cited. As subsequent processing, the system stores the generated summary data with an index in a dedicated table of the database, and makes it referable at high speed from a search API of a front end. The system adopts pipeline processing in which an extraction-type summary model and a generation-type summary model are connected in series, and generates a natural sentence while preventing information omission. With this configuration, the system realizes a technical improvement of completing reading and summarizing work of records, which takes several hours for a human, by computer processing of several seconds. As a result, the system exhibits a technical effect of significantly reducing latency of information retrieval and providing an environment in which the nursing care worker can instantaneously grasp the entire picture of the user. This technology is applicable to a legal tech field for analyzing an enormous number of past precedents, and a knowledge management system for summarizing a long-term project history.

Step 5: Secure information sharing, summarization, and anonymization by data linkage unit. The data linkage unit encrypts the nursing care record data recorded by the nursing care record management unit, and securely shares it with another nursing care facility or medical institution. At the time of transfer to another facility/hospital, administrative reporting, or linkage with a medical institution, a prompt sentence such as “Please summarize necessary information and output it in a form considering personal information protection in order to share a nursing care history of this user with another facility” is given to the generative AI, and summarization and anonymization of shared data are automatically performed so that only a destination having a decryption authority can view the content. Specifically, in this step, the system executes dynamic data masking and hybrid encryption processing based on a zero trust security model. The system receives, as input data, summarized nursing care record data to be shared and a public key certificate of a sharing destination system. As a specific example of the input data to the system, first, the summary text data of the user generated in Step 4 can be cited. Second, RSA 2048-bit public key data issued by the sharing destination medical institution can be cited. Third, a JSON object of an access control policy defining a range of data permitted to be shared can be cited. As preprocessing, the system physically deletes a field not permitted to be shared from the data set based on the access control policy. As output data, the system outputs payload data encrypted with a common key and common key data encrypted with a public key of the sharing destination. As a specific example of the output data, first, nursing care record data in a binary format encrypted by an AES256 algorithm can be cited. Second, digital envelope data including the encrypted common key can be cited. Third, a digital signature hash value for proving a transmission source of the data can be cited. As subsequent processing, the system combines these output data into one secure package, and transmits it to an API endpoint of the sharing destination system via a TLS protocol. The system forcibly executes a garbage collection process of securely erasing the encryption key and plaintext data on a memory after completion of the transmission. With this configuration, the system realizes a non-conventional secure communication method of automatically executing a series of processes from data anonymization to encryption and transmission without human intervention. As a result, the system exhibits a technical effect of mathematically eliminating a data leakage risk due to a man-in-the-middle attack or internal fraud, and establishing a secure data distribution infrastructure between organizations. This technology is applicable to a credit information sharing system between financial institutions and a confidential data linkage platform between companies in a supply chain.

Step 6: Falsification detection and abnormal pattern analysis. The falsification detection unit verifies the hash chain of the nursing care record data, and automatically checks whether a past record has been illegally rewritten. Furthermore, a prompt sentence such as “Please analyze in detail from a nursing care viewpoint whether there is an unnatural change or an abnormal pattern in this nursing care history, and point out a suspicious part if any” is given to the generative AI, and a pattern with a high possibility of falsification or fraud, such as duplicate records by a plurality of different staffs on the same day, a case where a plurality of records are collectively changed in a short period, or a case where there is a contradiction between care content and a user state, is automatically detected, and warning information is notified to an administrator or a nursing care worker. Specifically, in this step, the system executes data integrity verification applying a concept of blockchain technology and machine learning-based anomaly detection processing in parallel. The system receives, as input data, all log data sets in a period to be verified and a metadata feature amount of each log. As a specific example of the input data to the system, first, a chain array in which hash values of nursing care records for the past one month are connected can be cited. Second, a multidimensional vector including a creation time, an update time, and an IP address of a creation terminal of each record can be cited. Third, a chronological tensor of a behavior pattern extracted from a text of record content can be cited. As preprocessing, the system normalizes the chronological tensor and adapts it to the number of dimensions of an input layer of an anomaly detection model. As output data, the system outputs a continuity verification result of the hash chain and a score array indicating an anomaly probability of each log data. As a specific example of the output data, first, an array of Boolean values indicating that a recalculation result of the hash value matches a stored value can be cited. Second, a floating-point numerical value in a range of 0 to 1 indicating an anomaly degree of each log calculated by an isolation forest algorithm can be cited. Third, a contribution degree vector of a feature amount explaining a reason for being determined as an anomaly can be cited. As subsequent processing, when a mismatch of the hash chain is detected or when the anomaly score exceeds a threshold, the system moves the corresponding log to an isolation area. At the same time, the system establishes WebSocket communication for displaying an emergency alert on a dashboard of a system administrator. In learning of the anomaly detection model, the system adopts unsupervised learning using only past normal log data, and can cope with an unknown falsification method. With this configuration, the system specifies highly disguised unauthorized access or data falsification that cannot be found by a simple rule-based check by outlier detection in a high-dimensional space. As a result, the system exhibits a technical effect of dramatically improving a security level of the system and dramatically reducing time and cost required for audit response. This technology is applicable to an official document management system of a government agency and a transaction monitoring system of a virtual currency exchange.

### Specific Use Case

For example, a case is assumed where an elderly person requiring nursing care uses a plurality of nursing care facilities and receives a wide variety of care such as daily meal assistance, excretion assistance, bathing assistance, rehabilitation, medication management, and night monitoring. In this case, the nursing care record management unit records detailed care content input by a staff of each facility from a tablet terminal or a personal computer terminal, such as “breakfast assistance implemented, intake 80%, no choking”, “morning rehabilitation, walking training 20 minutes, walking distance 50 m, no fall”, “afternoon bathing assistance, no skin condition abnormality, monitoring when entering and exiting bathtub”, and “night excretion assistance twice, night awakening once, good sleep”, in chronological order together with user identification information, vital data, a cognitive function score, an ADL score, a fall history, a recording time, and a hash value of an immediately preceding record, and stores them as a hash chain. Furthermore, an accident/near-miss report, a medication error, a request or claim from a family member, a medical treatment, an outing/overnight stay record, an emergency transportation record, and the like are also recorded in detail, and data of a plurality of facilities are also integrated and managed. Specifically, in this specific example, the system executes data pipeline processing for integrating unstructured and semi-structured data generated from a heterogeneous distributed environment in real time. The system receives, as input data, data streams of various formats asynchronously transmitted from systems of a plurality of different nursing care facilities. As a specific example of the input data to the system, first, structured data in a JSON format including a meal intake amount and an excretion status transmitted from a terminal of a home-visit nursing care facility can be cited. Second, a numerical vector indicating a walking distance and an implementation time of rehabilitation acquired from a system of a day service center via an API can be cited. Third, chronological binary data indicating body movement on a bed or getting out of bed continuously transmitted from a night monitoring sensor by an MQTT protocol can be cited. As preprocessing, the system executes ETL processing for converting these data of different formats into a common internal schema. As output data, the system outputs an integrated data record normalized for each user and sorted in chronological order, and a cryptographic hash value assigned to each record. As a specific example of the output data, first, multidimensional array data in which care content of a plurality of facilities is mapped on a single timeline can be cited. Second, chain structure data of a SHA256 hash value for proving integrity of each record can be cited. Third, a metadata object including a facility ID and a terminal ID indicating a source of data can be cited. As subsequent processing, the system writes this integrated data record to a cluster of a distributed database with replication. When writing data, the system uses a conflict resolution algorithm to prevent data inconsistency due to simultaneous writing from a plurality of facilities. With this configuration, the system realizes a non-conventional data synchronization method of consistently integrating data generated from a plurality of physically separated systems as a single logical database. As a result, the system exhibits a technical effect of eliminating data silos and providing an integrated view capable of comprehensively grasping a user's state in real time. This technology is applicable to a regional medical information network in which a plurality of medical institutions and pharmacies cooperate, and a multi-sensor data integration infrastructure in a smart city.

The care plan AI determination unit gives a prompt sentence such as “Please explain in detail an optimal care plan for this user and a basis thereof from medical and nursing care viewpoints based on information such as an ADL score, a cognitive function score, a fall history, vital data, a medical history, a medication status, a rehabilitation progress, a family structure, and a living environment” to the generative AI, and automatically generates a complex care policy and a basis explanation, for example, “Since the ADL score is 20 points or less and full assistance is required, and the cognitive function score is 10 points or less and severe dementia is suspected, a two-caregiver system is recommended for all of meals, excretion, bathing, changing clothes, and transfer, and orientation training, environmental adjustment, and wandering countermeasures are enhanced as dementia care” and “Since there have been two falls in the past three months and many falls during night toilet movement, installation of night lighting, securing of a flow line to a toilet, introduction of a walking aid, and enhancement of a night monitoring system are recommended”. Specifically, in this specific example, the care plan AI determination unit executes an inference pipeline using a large language model capable of processing multimodal input data. The care plan AI determination unit receives, as input data, a feature tensor indicating a comprehensive state of the user extracted from the integrated database. As a specific example of the input data to the care plan AI determination unit, first, a normalized numerical vector indicating that the ADL score is 20 points or less and the cognitive function score is 10 points or less can be cited. Second, a chronological frequency array indicating a fall history for the past three months and the number of night toilet movements can be cited. Third, an embedding vector of text data describing a family structure and a living environment can be cited. As preprocessing, the care plan AI determination unit executes linear transformation processing for mapping these heterogeneous data to the same latent space. As output data, the care plan AI determination unit outputs a probability distribution for each action of the recommended care plan, and a natural language token sequence indicating a basis thereof. As a specific example of the output data, first, a score value indicating that a probability of recommending full assistance for meals and bathing is 98 percent can be cited. Second, a structured action list instructing installation of night lighting and introduction of a walking aid can be cited. Third, text data explaining a correlation between suspicion of severe dementia and a fall risk from a medical viewpoint can be cited. As subsequent processing, the care plan AI determination unit transmits the generated action list to an API of a task management system, and automatically assigns it to a business schedule of the nursing care staff. In the inference process, the care plan AI determination unit searches for a text sequence with the highest logical consistency using a beam search algorithm. With this configuration, the system instantaneously completes information collection and analysis work, which takes several hours for a human care manager, by vector calculation in a high-dimensional space. As a result, the system exhibits a technical effect of dramatically shortening a lead time for care plan creation, eliminating a personalized judgment error, and ensuring uniform care quality based on evidence. This technology is applicable to a delivery route optimization system for logistics that needs to satisfy complex constraint conditions, and an automatic formulation system for a preventive maintenance plan in a manufacturing industry.

The explanation generation unit gives prompt sentences such as “Please create an explanatory text that is easy for the user to understand based on this care plan and basis”, “Please explain including a care policy and life guidance in an expression easy for an elderly family member to understand”, and “Please explain including future precautions and monitoring points for a family member of a user with dementia” to the generative AI based on the care plan and the basis explanation generated by the care plan AI determination unit, and automatically generates an explanatory text, for example, “Since it is difficult for the user to perform activities of daily living, a caregiver will be attached to all of meals, bathing, excretion, changing clothes, and the like. While considering safety, we will also provide support to encourage independence as much as possible” and “Since a decline in cognitive function is observed, we will perform monitoring, calling out, and environmental adjustment so that the user can spend time with peace of mind. If wandering or confusion is observed, we will also thoroughly implement measures to prevent falls and accidents”. Specifically, in this specific example, the explanation generation unit executes a conditional language generation model that performs text style conversion according to an attribute of a target person. The explanation generation unit receives, as input data, professional care plan data output from the care plan AI determination unit and a profile vector of an explanation target person. As a specific example of the input data to the explanation generation unit, first, structured JSON data of a care plan including technical terms such as full assistance and environmental adjustment can be cited. Second, a numerical vector of age and IT literacy indicating that the explanation target person is an elderly family member can be cited. Third, a flag variable indicating that it is for the user with dementia himself/herself can be cited. As preprocessing, the explanation generation unit dynamically adjusts a temperature and a penalty coefficient, which are decoding parameters of a language model, based on the profile vector. As output data, the explanation generation unit outputs natural language text data optimized for the understanding level of the target person, and metadata including layout information for display. As a specific example of the output data, first, text data converted into a plain expression such as “We will help you with meals and bathing” avoiding technical terms can be cited. Second, a markup text in which specific precautions for fall prevention are itemized can be cited. Third, CSS style information for highlighting an important part of the text can be cited. As subsequent processing, the explanation generation unit transmits the generated text data to a push notification server of a smartphone application for family members. In learning of the model, the explanation generation unit implements supervised fine-tuning using pair data of plain explanatory texts created by an expert. With this configuration, the system realizes advanced semantic conversion processing considering a context and an attribute of a target person on a computer, rather than simple word replacement. As a result, the system exhibits a technical effect of reducing a risk of a claim or an accident due to a communication discrepancy, and improving satisfaction of the user and the family member and reliability of the system. This technology is applicable to a legal assistant AI that explains difficult legal documents to the general public, and a system that automatically generates a document explaining important matters of a financial product according to a customer's knowledge level.

The data linkage unit gives a prompt text such as “Please summarize necessary information and output it in a form considering personal information protection in order to share the nursing care history of this user with another office” to the generative AI at the time of transfer to another facility/hospital, administrative reporting, or linkage with a medical institution, summarizes main care content, an accident history, a rehabilitation progress, a medication status, a request history from a family member, and the like for the past one year, and encrypts and securely shares them. Specifically, in this specific example, the data linkage unit executes a secure pipeline combining context-aware data masking by natural language processing and hybrid encryption. The data linkage unit receives, as input data, all nursing care history data of a user to be shared and a query parameter indicating a requirement specification of a sharing destination. As specific examples of the input data to the data linkage unit, first, several megabytes of unstructured text data including care content and an accident history for the past one year can be cited. Second, a query vector in which the sharing destination is a medical institution and which requests extraction of a rehabilitation progress and a medication status can be cited. Third, a flag array of a security policy instructing complete anonymization of personal information can be cited. As preprocessing, the data linkage unit extracts information matching the requirement specification from the input text using a large language model, and simultaneously masks personal information such as a name and an address using a named entity recognition model. The data linkage unit outputs, as output data, text data that has been summarized and anonymized, and a ciphertext payload obtained by encrypting the data. As specific examples of the output data, first, summary text data obtained by compressing only main change points for the past one year into several hundred characters can be cited. Second, encrypted data in a binary format generated by an AES256 algorithm can be cited. Third, data of a session key encrypted with a public key of the sharing destination can be cited. As subsequent processing, the data linkage unit transmits the encrypted payload to a receiving endpoint of a sharing destination system via a RESTful API. The data linkage unit adopts a secure coding method of immediately discarding plaintext data on a memory in a processing process. With this configuration, the system solves conflicting requirements of protecting privacy while maintaining usefulness of data by a combination of advanced algorithms. As a result, the system exhibits a technical effect of enabling rapid and secure data linkage between organizations while minimizing an information leakage risk. This technology is applicable to a data room system for due diligence in M&A between companies and a traceability system of a supply chain shared by a plurality of companies.

The falsification detection unit verifies a hash chain of nursing care record data, gives a prompt text such as “Please analyze in detail from a nursing care perspective whether there is an unnatural change or an abnormal pattern in this nursing care history, and point out any suspicious part” to the generative AI, automatically detects a pattern with a high possibility of falsification or fraud, such as duplicate records by a plurality of different staff members on the same day, a case where a plurality of records are changed at once in a short period of time, or a case where there is a contradiction between care content and a user state, and notifies an administrator or a nursing care worker of warning information. Specifically, in this specific example, the falsification detection unit executes a dual-engine architecture integrating cryptographic verification and machine learning-based behavior detection. The falsification detection unit receives, as input data, a transaction log of a nursing care record periodically fetched from a database and a context feature amount of each log. As specific examples of the input data to the falsification detection unit, first, a blockchain-like data structure in which a hash value of each record is concatenated in chronological order can be cited. Second, a multidimensional vector of metadata including a creator ID of a record, an IP address of a terminal, and a stamping time can be cited. Third, a numerical tensor indicating recorded care content and a current ADL score of a user can be cited. As preprocessing, the falsification detection unit recalculates the hash chain and executes a bitwise comparison operation with a stored hash value at high speed. The falsification detection unit outputs, as output data, a status code indicating data integrity and an anomaly probability score output from an anomaly detection model. As specific examples of the output data, first, an error flag indicating a mismatch of hash values can be cited. Second, an anomaly score value of 0.85 indicating that duplicate records by a plurality of different staff members exist on the same day can be cited. Third, warning text data indicating a logical contradiction that a record of independent walking is input for a user requiring full assistance can be cited. As subsequent processing, the falsification detection unit saves a transaction whose anomaly score exceeds a threshold value to an isolation database, and transmits a push notification to a smartphone of the administrator. The falsification detection unit learns a chronological dependency of logs using a recurrent neural network, and detects a contextual anomaly that cannot be discriminated by a single log. With this configuration, the system realizes a non-conventional security method of automatically revealing an internal crime or advanced data falsification that cannot be prevented by simple access control through multifaceted data analysis. As a result, the system exhibits a technical effect of mathematically ensuring evidence capability of an electronic record and dramatically improving reliability of the system and transparency of an audit. This technology is applicable to an unauthorized remittance detection system of a financial institution and an automatic detection system of cheating in an online exam.

In this way, the system of the present invention centrally manages diverse nursing care records for each user in chronological order, integrally realizes optimal care plan determination and basis explanation by AI, automatic generation of an explanatory text for a user/family member, secure information sharing by summarization and anonymization, and falsification detection by a hash chain and AI analysis, and can significantly improve transparency, reliability, explainability, efficiency, and safety in a nursing care site. Specifically, the system functions as an integrated control platform that orchestrates a plurality of artificial intelligence models and encryption modules on a cloud-native microservice architecture infrastructure. The system receives, as input data, metadata of all transactions flowing through the entire system and telemetry data indicating an operating status of each microservice. As specific examples of the input data to the system, first, chronological numerical data indicating the number of API requests per second can be cited. Second, a floating-point array in milliseconds indicating an inference latency of each AI model can be cited. Third, a percentage value indicating a usage rate of a connection pool of a database can be cited. As preprocessing, the system inputs these telemetry data to a stream processing engine, and calculates statistics such as a moving average and a variance in real time. The system outputs, as output data, a control command instructing dynamic allocation of system resources and structured data for a dashboard indicating a performance index of the entire system. As specific examples of the output data, first, an autoscaling command for automatically adding a GPU node in response to an increase in inference load can be cited. Second, update data of a routing table for optimizing network traffic can be cited. Third, aggregated report data indicating that an availability of the entire system is 99.99 percent can be cited. As subsequent processing, the system transmits the control command to a control plane of a container orchestration tool to autonomously optimize a state of an infrastructure. With this configuration, the system realizes an autonomous control processing unique to a computer that maintains performance and stability of the entire system without human intervention even in an environment requiring enormous data processing and complex AI inference. As a result, the system exhibits a technical effect of always keeping a response speed to an end user optimal while maximizing utilization efficiency of hardware resources. This technology is widely applicable to a large-scale healthcare platform having millions of users and an integrated monitoring and control system of a mission-critical social infrastructure.

The specific processing unit 290 sends the results of specific processing obtained in Example 1 or Application Example 1 described above to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence).

An example of the data generation model 58 is a generative AI such as ChatGPT (registered trademark) (Internet search <URL: https://openai.com/blog/chatgpt>).

The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

In the above embodiments, an example form where specific processing is performed by the data processing device 12 was described, but the technology disclosed herein is not limited to this, and specific processing may be performed by the smart device 14.

### Second Embodiment

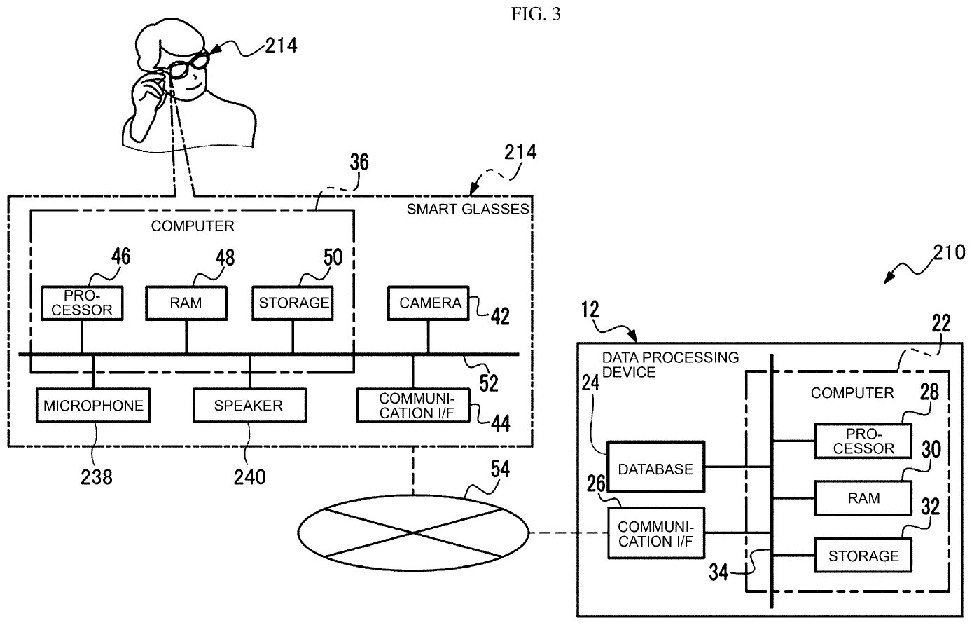

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, camera 42, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

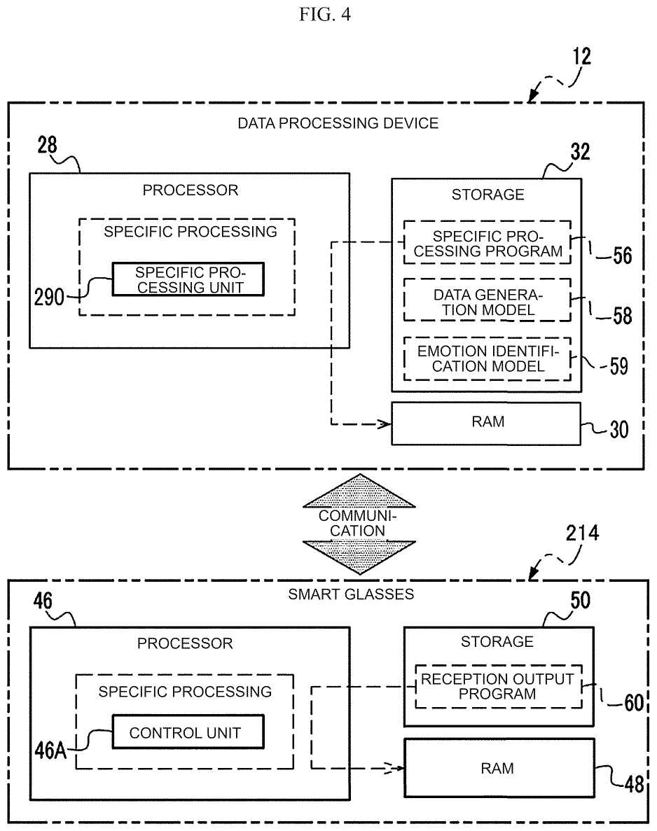

FIG. 4 shows an example of the main functions of the data processing device 12 and smart glasses 214. As shown in FIG. 4, specific processing is performed in the data processing device 12 by the processor 28. The storage 32 stores a specific processing program 56.

The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, and various estimations and predictions related to the user's emotions are performed, but it is not limited to such examples. Furthermore, emotion estimation and prediction may include, for example, emotion analysis.

In the smart glasses 214, reception output processing is performed by the processor 46. The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48. The smart glasses 214 may also have similar data generation models and emotion identification models as the data generation model 58 and emotion identification model 59, and perform the same processing as the specific processing unit 290 using these models.

Next, specific processing by the specific processing unit 290 of the data processing device 12 will be described. Each part of the system described below is realized by the data processing device 12 and the smart glasses 214. In the following description, the data processing device 12 is referred to as a “server,” and the smart glasses 214 are referred to as a “terminal.”

### Example 1

Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

In the above embodiments, an example form where specific processing is performed by the data processing device 12 was described, but the technology disclosed herein is not limited to this, and specific processing may be performed by the smart glasses 214.

### Third Embodiment

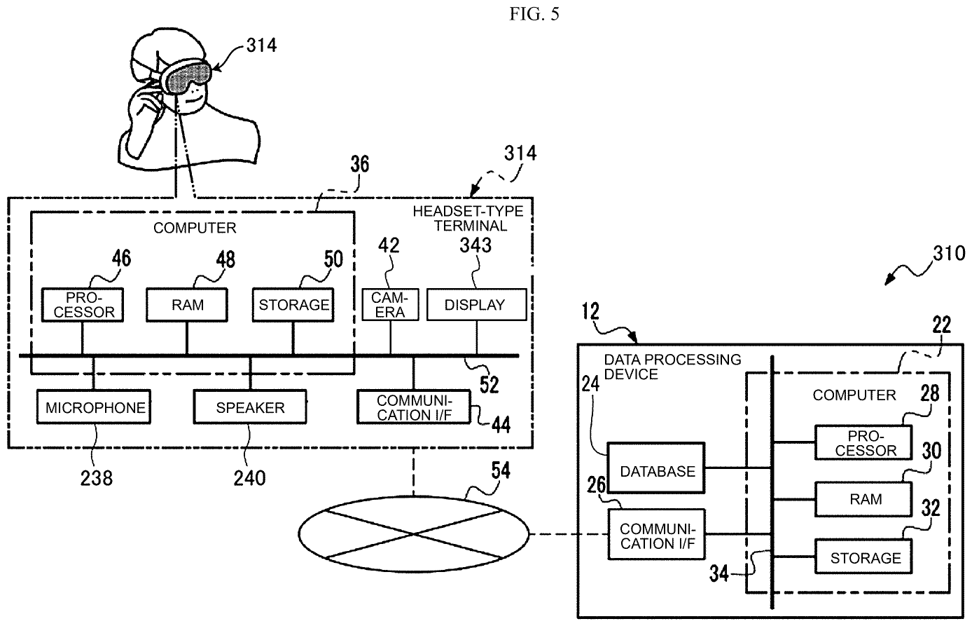

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, display 343, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

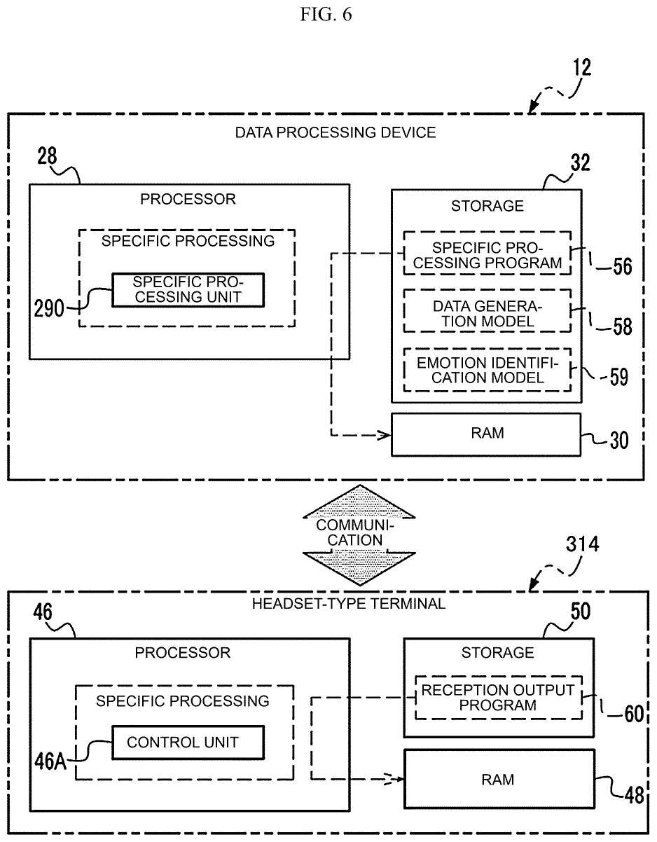

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290.

In the headset-type terminal 314, reception output processing is performed by the processor 46.

The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48.

Next, specific processing by the specific processing unit 290 of the data processing device 12 will be described. Each part of the system described below is realized by the data processing device 12 and the headset-type terminal 314. In the following description, the data processing device 12 is referred to as a “server,” and the headset-type terminal 314 is referred to as a “terminal.”

(Example 1) Since the flow of the specific processing is the same as that in Example 1 described in the first embodiment, the description thereof is omitted.

(Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

In the above embodiments, an example form where specific processing is performed by the data processing device 12 was described, but the technology disclosed herein is not limited to this, and specific processing may be performed by the headset-type terminal 314.

### Fourth Embodiment

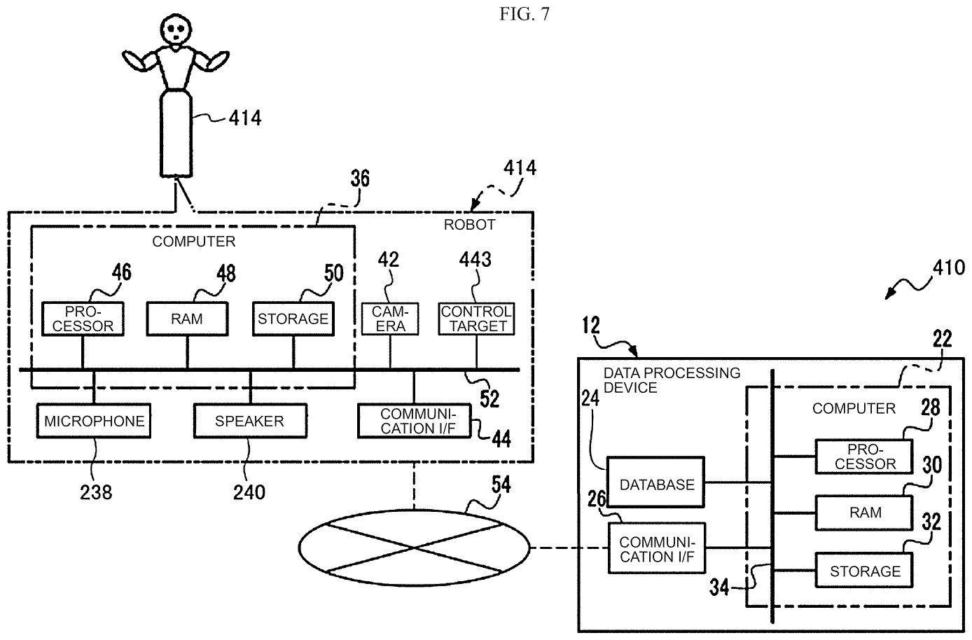

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 is an example of a “computer” related to the technology disclosed herein. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, control target 443, and communication I/F 44 are also connected to the bus 52.

The microphone 238 accepts voice uttered by the user 20, accepting instructions, among others, from the user 20. The microphone 238 captures the voice emitted by the user 20, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user 20 (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

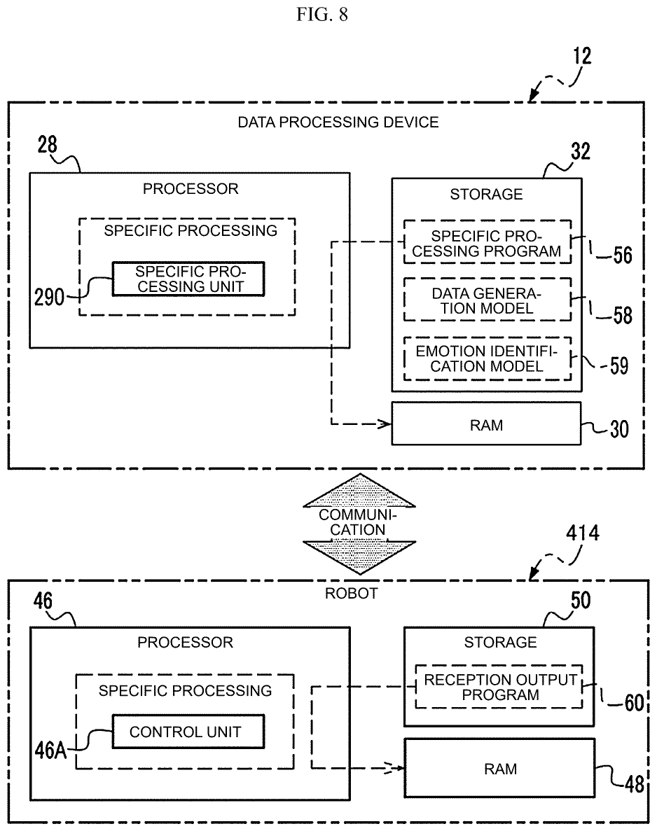

FIG. 8 shows an example of the main functions of the data processing device 12 and the robot 414. As shown in FIG. 8, specific processing is performed in the data processing device 12 by the processor 28. The storage 32 stores a specific processing program 56.

The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290.

In the robot 414, reception output processing is performed by the processor 46. The storage 50 stores a reception output program 60. The processor 46 reads the reception output program 60 from the storage 50 and executes it on the RAM 48. The reception output processing is realized by the processor 46 operating as a control unit 46A according to the reception output program 60 executed on the RAM 48.

Next, specific processing by the specific processing unit 290 of the data processing device 12 will be described. Each part of the system described below is realized by the data processing device 12 and the robot 414. In the following description, the data processing device 12 is referred to as a “server,” and the robot 414 is referred to as a “terminal.” (Example 1) Since the flow of the specific processing is the same as that in Example 1 described in the first embodiment, the description thereof is omitted. (Application Example 1) Since the flow of the specific processing is the same as that in Application Example 1 described in the first embodiment, the description thereof is omitted.

The specific processing unit 290 sends the results of specific processing obtained in Example 1 or Application Example 1 described above to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

Moreover, the processing by the data processing system 10 described above is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

In the above embodiments, an example form where specific processing is performed by the data processing device 12 was described, but the technology disclosed herein is not limited to this, and specific processing may be performed by the robot 414.

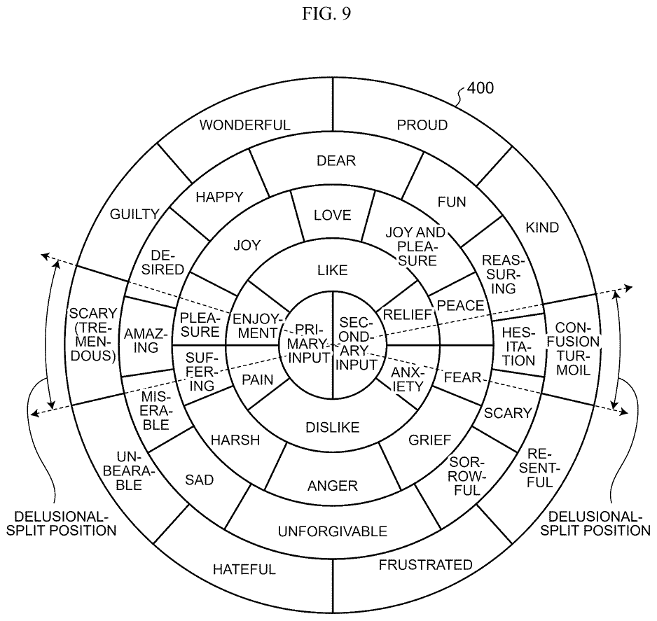

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

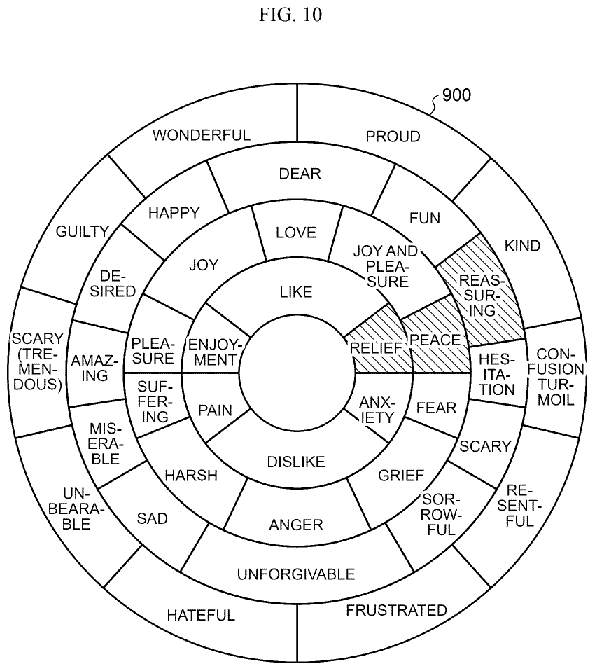

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above, the system according to the present disclosure has been described mainly on the functions of the data processing device 12, but the system according to the present disclosure is not necessarily mounted on a server. The system according to the present disclosure may be implemented as a general information processing system. The present disclosure may be implemented, for example, as a software program running on a personal computer, or an application running on a smartphone or the like. The method according to the present disclosure may be provided to the user in the form of SaaS (Software as a Service).

In the above embodiments, an example form where specific processing is performed by a single computer 22 was described, but the technology disclosed herein is not limited to this, and distributed processing for specific processing by multiple computers including the computer 22 may be performed. For example, the data generation model 58 may be provided in an external device of the data processing device 12, and the external device may generate data corresponding to the input data.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned.

Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

Note that the following supplementary notes are further disclosed regarding the above description.

### Supplementary Note 1

A system comprising: a nursing care record management unit; a care plan AI determination unit; an explanation generation unit; a data linkage unit; and a falsification detection unit, wherein the nursing care record management unit is configured to record nursing care record data for each user in chronological order, assign user identification information, care content, vital data, a cognitive function score, an ADL score, a fall history, a recording time, and a hash value of an immediately preceding record to each nursing care record, and store the nursing care record as a hash chain; the care plan AI determination unit is configured to automatically determine an optimal care plan based on the nursing care record data using generative AI, and explicitly generate a data item, a selection reason, and a medical/nursing care basis used for the care plan determination as an explanatory text; the explanation generation unit is configured to automatically generate an explanatory text in a natural language easy for a user or a family member to understand using generative AI based on the care plan and the basis explanation generated by the care plan AI determination unit; the data linkage unit is configured to encrypt the nursing care record data recorded by the nursing care record management unit to enable secure sharing with another nursing care facility or medical institution, and enable a receiving side to decrypt the nursing care record data to view a nursing care history; and the falsification detection unit is configured to automatically detect presence or absence of falsification of the nursing care record data by verifying the hash chain, and these units cooperate to realize transparency, reliability, explainability, and secure sharing of the nursing care record data.

### Supplementary Note 2

The system according to Supplementary Note 1, wherein the care plan AI determination unit is configured to automatically generate a summary of the nursing care record data of the user using generative AI to enable a nursing care worker to grasp the entire nursing care history of the user in a short time, and the summary information is also recorded as a part of the hash chain.

### Supplementary Note 3

The system according to Supplementary Note 1, wherein, in addition to the hash chain verification of the nursing care record data, the falsification detection unit is configured to analyze whether an unnatural change or an abnormal pattern is included in the content of the nursing care record data using generative AI, and automatically generate warning information to notify an administrator or a nursing care worker when there is a possibility of falsification or fraud.

What is claimed is:

1. A system comprising:
circuitry configured to:
receive, via a communication interface coupled to a packet-switched network, unstructured data records from a plurality of data source endpoints, and store the unstructured data records in a storage device;
apply a natural language processing model to the unstructured data records to extract structured attribute fields, generate record embedding vectors, and index the record embedding vectors in a vector database;
apply a hash function to each unstructured data record entry and link successive hash values to form a tamper-resistant append-only log in the storage device, and verify data integrity by recomputing hash values across stored records at configurable intervals;
apply a generative neural network model to a query embedding vector derived from a received query and context records retrieved from the vector database by cosine similarity search to generate an inference output record and an attribution vector comprising source document identifiers; and
transmit the inference output record, the attribution vector, and a hash chain verification status derived from the tamper-resistant append-only log to terminal devices via the communication interface.

2. The system according to claim 1, wherein the circuitry is configured to apply a named entity recognition model to the unstructured data records to extract entity labels comprising event type identifiers, timestamp fields, and responsible party identifiers, normalize the extracted fields to a unified schema, and store the normalized attribute fields as structured records in the storage device.

3. The system according to claim 2, wherein the circuitry is configured to apply the natural language processing model to compute term frequency-inverse document frequency scores for key phrase extraction, generate phrase-level embedding vectors from the extracted key phrases, and incorporate the phrase-level embedding vectors as additional index entries in the vector database.

4. The system according to claim 1, wherein the circuitry is configured to generate the query embedding vector by applying the natural language processing model to query text received from a terminal device, compute cosine similarity scores between the query embedding vector and record embedding vectors indexed in the vector database, and select records having cosine similarity scores exceeding a retrieval threshold parameter.

5. The system according to claim 4, wherein the circuitry is configured to apply a cross-encoder relevance model to rerank the selected records by jointly encoding the query text and each candidate record to compute a refined relevance score, and incorporate the top-ranked records weighted by relevance score as context in the generative neural network model prompt.

6. The system according to claim 1, wherein the circuitry is configured to apply a multi-stage retrieval process by first selecting candidate records using approximate nearest neighbor indexing over the vector database, then reranking using the cross-encoder relevance model, and generating the inference output record by autoregressive decoding conditioned on the reranked context records.

7. The system according to claim 6, wherein the circuitry is configured to generate the attribution vector by extracting source document identifiers from the context records incorporated in the generation prompt, compute a contribution score for each source document by measuring the overlap between the inference output record and the corresponding context record content, and rank source documents in descending order of contribution score.

8. The system according to claim 1, wherein the circuitry is configured to apply the hash function sequentially to each new unstructured data record entry by computing a cryptographic hash of the record content concatenated with the hash value of the preceding entry, store each hash value with its corresponding record identifier and timestamp in the tamper-resistant append-only log, and detect integrity violations by identifying mismatches between stored and recomputed hash values.

9. The system according to claim 8, wherein the circuitry is configured to generate the hash chain verification status by scanning the tamper-resistant append-only log for hash value mismatches, compute a chain integrity score representing the proportion of verified entries, and append the chain integrity score and a list of unverified record identifiers to the hash chain verification status transmitted to terminal devices.

10. The system according to claim 1, wherein the circuitry is configured to monitor external data service endpoints via periodic polling to detect new unstructured data records, apply the natural language processing model to generate updated record embedding vectors for newly detected records, and insert the updated embedding vectors into the vector database index.

11. The system according to claim 10, wherein the circuitry is configured to detect semantic overlap between new record embedding vectors and existing record embedding vectors using cosine similarity scoring, generate a deduplication flag when similarity exceeds a deduplication threshold, and route flagged records to a manual review queue prior to indexing.

12. The system according to claim 1, wherein the circuitry is configured to generate a confidence score for the inference output record by applying a factual grounding model that computes the degree of alignment between the inference output record content and the content of the retrieved context records, and append the confidence score to the inference output record transmitted to terminal devices.

13. The system according to claim 12, wherein the circuitry is configured to route inference output records having confidence scores below a confidence threshold to a human review queue, generate a partial draft record comprising the inference output record and the attribution vector, and transmit the partial draft record to a terminal device associated with a reviewer user identifier.

14. The system according to claim 1, wherein the circuitry is configured to apply a change detection algorithm to updated unstructured data records by computing cosine similarity between updated record embedding vectors and existing embedding vectors for matching record identifiers, trigger a re-indexing operation when similarity falls below a change detection threshold, and update the tamper-resistant append-only log with a hash entry for each updated record.

15. The system according to claim 1, wherein the circuitry is configured to enforce a role-based access control policy by associating record categories in the vector database with required user role parameters, and restrict retrieval to records within the permission scope of the requesting terminal device user role parameter.

16. The system according to claim 1, wherein the circuitry is configured to update the generative neural network model by generating training pairs from historical query embedding vectors and corresponding validated inference output records, and fine-tune the model to improve output relevance and attribution accuracy for domain-specific query patterns.

17. The system according to claim 1, wherein the unstructured data records are received from domain-specific data source endpoints and comprise time-stamped event records and associated contextual data, the tamper-resistant append-only log provides an auditable chain of record provenance, and the attribution vector identifies the source records contributing to each inference output record.

18. A system comprising:
circuitry configured to:
receive, via a communication interface coupled to a packet-switched network, unstructured data records from data source endpoints, and apply a natural language processing model to extract structured attribute fields and generate record embedding vectors indexed in a vector database;
apply a hash function to each data record entry to form a tamper-resistant append-only log, and verify data integrity by recomputing hash values at configurable intervals;
apply a generative neural network model to a query embedding vector and context records retrieved by cosine similarity to generate an inference output record and an attribution vector comprising source document identifiers; and
transmit the inference output record, the attribution vector, and a hash chain verification status to terminal devices via the communication interface.

19. The system according to claim 18, wherein the circuitry is configured to apply a cross-encoder relevance model to rerank retrieved context records by refined relevance scores, generate a confidence score by applying a factual grounding model to measure alignment between the inference output record and retrieved context records, and route low-confidence records to a human review queue with a partial draft for reviewer terminal devices.

20. A method comprising:
receiving, via a communication interface coupled to a packet-switched network, unstructured data records from a plurality of data source endpoints, and storing the unstructured data records in a storage device;
applying a natural language processing model to the unstructured data records to extract structured attribute fields, generate record embedding vectors, and index the record embedding vectors in a vector database;
applying a hash function to each unstructured data record entry and linking successive hash values to form a tamper-resistant append-only log, and verifying data integrity by recomputing hash values at configurable intervals;
applying a generative neural network model to a query embedding vector and context records retrieved from the vector database by cosine similarity search to generate an inference output record and an attribution vector comprising source document identifiers; and
transmitting the inference output record, the attribution vector, and a hash chain verification status to terminal devices via the communication interface.
